SEQUENCE LISTING (23 sequences)

SEQ ID NO: 1 - protein, 23 aa, synthetic construct
  source 1..23: note = HER2 STMD
SIISAVVGILLVVVLGVVFGILI

SEQ ID NO: 2 - protein, 119 aa, synthetic construct
  source 1..119: note = anti-HER2 Variable Heavy domain of Heavy chain (VHH) nanobody
ESGGGSVQSGGSLRLSCAASGYNFGWYCMGWFRQAPGKEREGVASIGGSSITKYSDSVKGRFTISRDNAKNTLYLQMNALKPEDAATYYCAARPEYDCDSLREAGWRYWGQGTQVTVSS

SEQ ID NO: 3 - protein, 124 aa, synthetic construct
  source 1..124: note = anti-EGFR
EVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 4 - protein, 130 aa, synthetic construct
  source 1..130: note = anti-EpCAM
QVQLVQSGGGSVQGGASLRLSCAASGGERNNYCVAWFRQAPGKEREVAAISRAASGAQTTTKYYVDSVKGRFTISQDTKNTATVYLQMNSHKPEDTAYCTAKAKIYPPQCTGISRTIDYRGQGTQVTVSS

SEQ ID NO: 5 - protein, 10 aa, synthetic construct
  source 1..10: note = cysteine conjugated through post-translational modifications, with palmitic acid, and a palmitoylation sequence
MLCCMRRTKQ

SEQ ID NO: 6 - protein, 6 aa, synthetic construct
  source 1..6: note = Hexahistidine tag
HHHHHH

SEQ ID NO: 7 - protein, 5 aa, synthetic construct
  source 1..5: note = GGGGS flexible linker
GGGGS

SEQ ID NO: 8 - protein, 6 aa, synthetic construct
  source 1..6: note = mysistolylation signal
MGSSKS

SEQ ID NO: 9 - protein, 5 aa, synthetic construct
  source 1..5: note = Rigid linker
EAAAK

SEQ ID NO: 10 - protein, 188 aa, synthetic construct
  source 1..188: note = anti-HER2 NB with a linker and a hexahistidine tag
HHHHHHESGGGSVQSGGSLRLSCAASGYNFGWYCMGWFRQAPGKEREGVASIGGSSITKYSDSVKGRFTISRDNAKNTLYLQMNALKPEDAATYYCAARPEYDCDSLREAGWRYWGQGTQVTVSSGGGGSGGGGSGGGGSGGGGSGGGGSGGGGSGGGGSGGGGSSIISAVVGILLVVVLGVVFGILI

SEQ ID NO: 11 - protein, 176 aa, synthetic construct
  source 1..176: note = anti-HER2 NB with a linker and a hexahistidine tag and a palmitic tail
HHHHHHESGGGSVQSGGSLRLSCAASGYNFGWYCMGWFRQAPGKEREGVASIGGSSITKYSDSVKGRFTISRDNAKNTLYLQMNALKPEDAATYYCAARPEYDCDSLREAGWRYWGQGTQVTVSSGGGGSGGGGSGGGGSGGGGSGGGGSGGGGSGGGGSGGGGSCMLCCMRRTKQ

SEQ ID NO: 12 - protein, 161 aa, synthetic construct
  source 1..161: note = Anti-EGFR NB with a hexahistidine tag, and a palmitic tail, and a linker
HHHHHHEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSGGGGSCMLCCMRRTKQ

SEQ ID NO: 13 - protein, 171 aa, synthetic construct
  source 1..171: note = Anti-EGFR NB with a hexahistidine tag and a palmitic tail, and a linker
HHHHHHEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSGGGGSGGGGSGGGGSCMLCCMRRTKQ

SEQ ID NO: 14 - protein, 181 aa, synthetic construct
  source 1..181: note = Anti-EGFR NB with a hexahistidine tag and a palmitic tail, and a linker
HHHHHHEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSGGGGSGGGGSGGGGSGGGGSGGGGSCMLCCMRRTKQ

SEQ ID NO: 15 - protein, 167 aa, synthetic construct
  source 1..167: note = Anti-EpCAM NB with a hexahistidine tag, a palmitic tail, and a linker
HHHHHHQVQLVQSGGGSVQGGASLRLSCAASGGERNNYCVAWFRQAPGKEREVAAISRAASGAQTTTKYYVDSVKGRFTISQDTKNTATVYLQMNSHKPEDTAYCTAKAKIYPPQCTGISRTIDYRGQGTQVTVSSGGGGSGGGGSGGGGSGGGGSCMLCCMRRTKQ

SEQ ID NO: 16 - protein, 177 aa, synthetic construct
  source 1..177: note = Anti-EpCAM NB with a hexahistidine tag, a palmitic tail, and a linker
HHHHHHQVQLVQSGGGSVQGGASLRLSCAASGGERNNYCVAWFRQAPGKEREVAAISRAASGAQTTTKYYVDSVKGRFTISQDTKNTATVYLQMNSHKPEDTAYCTAKAKIYPPQCTGISRTIDYRGQGTQVTVSSGGGGSGGGGSGGGGSGGGGSGGGGSGGGGSCMLCCMRRTKQ

SEQ ID NO: 17 - protein, 187 aa, synthetic construct
  source 1..187: note = Anti-EpCAM NB with a palmitic tail, a hexahistidine tag, and a linker
HHHHHHQVQLVQSGGGSVQGGASLRLSCAASGGERNNYCVAWFRQAPGKEREVAAISRAASGAQTTTKYYVDSVKGRFTISQDTKNTATVYLQMNSHKPEDTAYCTAKAKIYPPQCTGISRTIDYRGQGTQVTVSSGGGGSGGGGSGGGGSGGGGSGGGGSGGGGSGGGGSGGGGSCMLCCMRRTKQ

SEQ ID NO: 18 - protein, 156 aa, synthetic construct
  source 1..156: note = Anti-EGFR NB with a myristic tail, and (GGGGS)x4
MGSSKSGGGGSGGGGSGGGGSGGGGSHHHHHHEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 19 - protein, 156 aa, synthetic construct
  source 1..156: note = Anti-EGFR NB with a myristic tail, and linker (GGGGS)x1(EAAAK)x3
MGSSKSGGGGSEAAAKEAAAKEAAAKHHHHHHEVQLVESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 20 - protein, 162 aa, synthetic construct
  source 1..162: note = Anti-EpCAM NB with a myristic tail and linker (GGGGS)x4
MGSSKSGGGGSGGGGSGGGGSGGGGSHHHHHHQVQLVQSGGGSVQGGASLRLSCAASGGERNNYCVAWFRQAPGKEREVAAISRAASGAQTTTKYYVDSVKGRFTISQDTKNTATVYLQMNSHKPEDTAYCTAKAKIYPPQCTGISRTIDYRGQGTQVTVSS

SEQ ID NO: 21 - protein, 162 aa, synthetic construct
  source 1..162: note = Anti-EpCAM NB with a myristic tail (GGGGS)x1(EAAAK)x3
MGSSKSGGGGSEAAAKEAAAKEAAAKHHHHHHQVQLVQSGGGSVQGGASLRLSCAASGGERNNYCVAWFRQAPGKEREVAAISRAASGAQTTTKYYVDSVKGRFTISQDTKNTATVYLQMNSHKPEDTAYCTAKAKIYPPQCTGISRTIDYRGQGTQVTVSS

SEQ ID NO: 22 - protein, 11 aa, synthetic construct
  source 1..11: note = Palmitoylation inducer
CMLCCMRRTKQ

SEQ ID NO: 23 - protein, 10 aa, synthetic construct
  source 1..10: note = Semi-flexible linker
GGGGSEAAAK

(19) United States
(12) Patent Application Publication
(10) Pub. No.: US 2026/0250417 A1
(43) Pub. Date: Aug. 27, 2026

(54) CHIMERIC NANOBODY-DECORATED LIPOSOMES BY SELF-ASSEMBLY

(71) Applicant: The Research Foundation for The State University of New York, Binghamton, NY (US)

(72) Inventors: Yuan Wan, Binghamton, NY (US); Mofizur Rahman, Vestal, NY (US)

(21) Appl. No.: 19/052,232

(22) Filed: Feb. 12, 2025

Related U.S. Application Data
(60) Provisional application No. 63/522,527, filed on Jun. 22, 2023.

(51) Int. Cl. C07K 16/32; A61K 47/68; A61K 47/69; C07K 14/71; C07K 16/28; C07K 16/30

(52) U.S. Cl. CPC C07K 16/32; A61K 47/6849; A61K 47/6855; A61K 47/6911; C07K 14/71; C07K 16/2863; C07K 16/30; C07K 2317/569; C07K 2319/90; C07K 2319/915

(57) ABSTRACT

An engineered nanobody conjugate is provided, comprising: a Variable Heavy domain of Heavy chain (VHH) nanobody; a peptide single transmembrane domain (STMD); and a peptide linker between the nanobody and the STMD. The invention also provides a method of making an immunoliposome composition, comprising: mixing the engineered nanobody according to claim 1 with a liposome composition; and forming 100 nm liposomes from the liposome composition, having between 200 to 2500 chimeric nanobodies per 100 nm liposome. An engineered nanobody conjugate is provided comprising an ordered sequence of a peptide affinity tag, a Variable Heavy domain of Heavy chain (VHH) nanobody, a peptide linker, a cysteine residue, and a peptide fatty acylation signal.

### CROSS REFERENCE TO RELATED APPLICATIONS

The present application is a non-provisional of, and claims priority under 35 U.S.C. § 119(e) from, U.S. Provisional Patent Application No. 63/552,527, filed Feb. 12, 2024, the entirety of which is expressly incorporated herein by reference.

### INCORPORATION BY REFERENCE OF A SEQUENCE LISTING XML

A Sequence Listing is provided herewith as a Sequence Listing XML, “SUNY-RB-722.1.xml” created on May 5, 2026 and having a size of 28,137 bytes. The contents of the Sequence Listing XML are incorporated by reference herein in their entirety.

### FIELD OF THE INVENTION

The present invention relates to the field of targeted cancer therapy, and in particular a chimeric nanobody decorated liposome.

### BACKGROUND OF THE INVENTION

Targeting moieties grafted onto liposomes (LP), known as immunoliposomes (iLP), offer altered pharmacokinetics, improved drug tolerance, and enhance treatment efficacy compared to conventional LPs. However, due to the dysfunction of targeting moieties and payload loss during preparation, iLPs have yet to be favored in commercial manufacturing. On the other hand, tumor-targeting mRNA-lipid nanoparticles (LNP), which induce selective tumor cell immune elimination while minimizing damage to healthy tissues, face the same manufacturing challenges as iLPs. In addition, the use of polyethylene glycol (PEG) in iLP and LNP formulations may induce unintended immune responses. PEG is widely used to stabilize LPs, iLPs, and LNPs and prolong their circulation half-life by forming a hydrated sheath. However, a PEG component can lead to the production of anti-PEG antibodies or activate complement system, which can diminish therapeutic effectiveness, accelerate blood clearance, or trigger allergic reactions. Accordingly, FDA now requires monitoring of anti-PEG antibody responses in new PEG-containing drugs. Novel approaches to mitigate immunogenicity of PEG may be required. Notably, current exploration of PEG alternatives indicates that a protein sheath as an outer layer could enhance stability, half-life, and biocompatibility of these nanocarriers.

In summary, the existing preparation procedure for iLPs and tumor-targeting mRNA-LNPs involves laborious chemical modification, compromising the stability of targeting moieties. Payload leakage and product loss are also inevitable during the lengthy production process. Despite current efforts, these challenges persist, and iLPs and tumor-targeting mRNA-LNPs remain unappealing to manufacturers due to the high production cost, unscalable manufacturing, and batch-to-batch variation.

To facilitate the manufacturing of iLPs and tumor-targeting mRNA-LNPs, chimeric nanobodies (cNBs) have been developed (FIG. 1A). Produced in cell factories (either bacterial or mammalian cells), cNBs consist of a nanobody (NB) head targeting tumor-specific antigens, a flexible peptide linker, and a transmembrane segment (either a single transmembrane peptide or a lipid acid). In an optimized ratio, lipids, payloads, and cNBs self-assemble into iLPs or tumor-targeting LNPs in a single step (FIG. 1B). The method enables iLP and mRNA-LNP production using the existing manufacturing pipeline, showing promise for industrial manufacturing and clinical use.

Liposomes are one of the most successful drug delivery systems, with inherent advantages including self-assembly, large payload capacity, altered pharmacokinetics, and reduced systemic toxicity of entrapped drugs. Tremendous optimization work has been made on liposomes to improve stability, biocompatibility, and treatment efficacy. These efforts have led to successful clinical applications of liposomes. Notably, commercialized liposomal formulations rely mainly on passive targeting, i.e., enhanced permeability retention, which improves drug tolerance but fails to achieve proven effectiveness in the commercially desirable solid tumor setting. To achieve lesion-specific distribution, targeting moieties have been added to the liposomal surface to direct their delivery, improve tissue permeation, and enhance local accumulation. While actively targeted liposomes have been well demonstrated in laboratories, the industrial translation is partially hampered by the unappealing production process, such as laborious chemical modification, impaired targeting moieties, and payload loss (FIG. 1A).

In recent studies, targeting moieties were pre-conjugated with lipids and then introduced to liposomal membranes via lipid self-assembly, which is accompanied with drug encapsulation. Alternatively, the Fc-binding ligands are conjugated to liposomal membranes followed by the introduction of targeting moieties. These improvements increase production efficiency and quality to a certain degree. However, the lipidation of targeting moieties or Fc-binding ligands still requires chemical conjugation, capping reaction, and compulsory purification to remove chemical residues and excess targeting moieties. In addition, the full-length antibody has poor tissue penetration in solid tumors partly due to their large size and high affinity, which limits the therapeutic efficacy of antibody-functionalized liposomes. Although full antibody fragments and aptamers show improved tissue penetration, their low stability to thermal stress, low pH, and high ionic strength may impair their performance. In comparison, a nanobody (NB) with a small size has decent stability and low immunogenicity, which is a good candidate for preparing immunoliposomes.
Abdolvahab, Mohadeseh Haji, Pegah Karimi, Nasrin Mohajeri, Mohammad Abedini, and Hamed Zare. “Targeted drug delivery using nanobodies to deliver effective molecules to breast cancer cells: the most attractive application of nanobodies.” Cancer Cell International 24, no. 1 (2024): 67.
Abraham M J, Murtola T, Schulz R, Pill S, Smith J C, Hess B, et al. GROMACS: High performance molecular simulations through multi-level parallelism from laptops to supercomputers. SoftwareX 2015, 1-2: 19-25.
Agrawal, H., Zelisko, M., Liu, L. P. & Sharma, P. Rigid proteins and softening of biological membranes—with application to HIV-induced cell membrane softening. Sci Rep-Uk 6, doi:ARTN 25412 10.1038/srep25412 (2016).
Alavi, M. & Hamidi, M. Passive and active targeting in cancer therapy by liposomes and lipid nanoparticles. Drug Metab Pers Ther 34, doi:10.1515/dmpt-2018-0032 (2019).
Allen T M, Cullis P R. Drug delivery systems: entering the mainstream. Science 2004, 303(5665): 1818-1822.
Andersen T, Bleher S, Eide Flaten G, Tho I, Mattson S, Skalk0-Basnet N. Chitosan in mucoadhesive drug delivery: focus on local vaginal therapy. Mar Drugs 2015, 13(1): 222-236.
Barenholz Y. Doxil (R)—The first FDA-approved nano-drug: Lessons learned. Journal of Controlled Release 2012, 160(2): 117-134.
Bouma, R. G., M K Nijen Twilhaar, H. J. Brink, A. J. Affandi, B. S. Mesquita, K. Olesek, J. M. A. van Dommelen et al. “Nanobody-liposomes as novel cancer vaccine platform to efficiently stimulate T cell immunity.” International Journal of Pharmaceutics 660 (2024): 124254.
Boutin, J. A. Myristoylation. Cell Signal 9, 15-35 (1997).
Bragin P E, Mineev K S, Bocharova O V, Volynsky P E, Bocharov E V, Arseniev A S. HER2 Transmembrane Domain Dimerization Coupled with Self-Association of Membrane-Embedded Cytoplasmic Juxtamembrane Regions. J Mol Biol 2016, 428(1): 52-61.
Callister, W. D. & Rethwisch, D. G. Materials science and engineering: an introduction, Vol. 7. (Wiley New York, 2020).
Castorph S, Riedel D, Arleth L, Sztucki M, Jahn R, Holt M, et al. Structure parameters of synaptic vesicles quantified by small-angle x-ray scattering. Biophys J 2010, 98(7): 1200-1208.
Chauhan V P, Jain R K. Strategies for advancing cancer nanomedicine. Nat Mater 2013, 12(11): 958-962.
Chen X, Zaro J L, Shen W C. Fusion protein linkers: property, design and functionality. Adv Drug Deliv Rev 2013, 65(10): 1357-1369.
Chen Z, Moon J J, Cheng W. Quantitation and Stability of Protein Conjugation on Liposomes for Controlled Density of Surface Epitopes. Bioconjug Chem 2018, 29(4): 1251-1260.
Chen, B. M., Cheng, T. L. & Roffler, S. R. Polyethylene Glycol Immunogenicity: Theoretical, Clinical, and Practical Aspects of Anti-Polyethylene Glycol Antibodies. Acs Nano 15, 14022-14048 (2021).
Chen, X., Zaro, J. L. & Shen, W. C. Fusion protein linkers: property, design and functionality. Adv Drug Deliv Rev 65, 1357-1369, doi:10.1016/j.addr.2012.09.039 (2013).
Chen, Z., Moon, J. J. & Cheng, W. Quantitation and Stability of Protein Conjugation on Liposomes for Controlled Density of Surface Epitopes. Bioconjug Chem 29, 1251-1260, doi:10.1021/acs.bioconjchem.8b00033 (2018).
Cheng, X. & Lee, R. J. The role of helper lipids in lipid nanoparticles (LNPs) designed for oligonucleotide delivery. Adv Drug Deliv Rev 99, 129-137 (2016).
Cho E J, Lee J W, Ellington A D. Applications of Aptamers as Sensors. Annu Rev Anal Chem 2009, 2: 241-264.
Chrai, S. S., Murari, R. & Ahmad, I. Liposomes (a review)—Part two: Drug delivery systems. Biopharm-Appl T Bio 15, 40-+(2002).
Custodio T F, Das H, Sheward D J, Hanke L, Pazicky S, Pieprzyk J, et al. Selection, biophysical and structural analysis of synthetic nanobodies that effectively neutralize SARS-CoV-2. Nature Communications 2020, 1 1(1).
Dammer U, Hegner M, Anselmetti D, Wagner P, Dreier M, Huber W, et al. Specific antigen/antibody interactions measured by force microscopy. Biophys J 1996, 70(5): 2437-2441.
Dammes, N., et al. Conformation-sensitive targeting of lipid nanoparticles for RNA therapeutics. Nat Nanotechnol 16, 1030-+(2021).
De Meyer, T., Muyldermans, S. & Depicker, A. Nanobody-based products as research and diagnostic tools. Trends Biotechnol 32, 263-270, doi:10.1016/j.tibtech.2014.03.001 (2014).
Decuzzi P, Ferrari M. The adhesive strength of non-spherical particles mediated by specific interactions. Biomaterials 2006, 27(30): 5307-5314.
DeLano, W. L. PyMOL molecular viewer: Updates and refinements. Abstr Pap Am Chem S 238 (2009).
Dhritlahre, Rakesh Kumar, and Ankit Saneja. “Recent advances in HER2-targeted delivery for cancer therapy.” Drug discovery today 26, no. 5 (2021): 1319-1329.
Dokukin M E, Sokolov I. Quantitative mapping of the elastic modulus of soft materials with HarmoniX and PeakForce QNM AFM modes. Langmuir 2012, 28(46): 16060-16071.
Dominguez L, Foster L, Straub J E, Thirumalai D. Impact of membrane lipid composition on the structure and stability of the transmembrane domain of amyloid precursor protein. P Nat/Acad Sci 459 USA 2016, 113(36): E5281-E5287.
Dostalova S, Vasickova K, Hynek D, Krizkova S, Richtera L, Vaculovicova M, et al. Apoferritin as an ubiquitous nanocarrier with excellent shelf life. Int J Nanomed 2017, 12: 2265-2278.
Dunphy, J. T. & Linder, M. E. Signalling functions of protein palmitoylation. Biochim Biophys Acta 1436, 245-261 (1998).
Et-Thakafy O, Delorme N, Gaillard C, Meriadec C, Artzner F, Lopez C, et al. Mechanical Properties of Membranes Composed of Gel-Phase or Fluid-Phase Phospholipids Probed on Liposomes by Atomic Force Spectroscopy. Langmuir 2017, 33(21 51 17-5126.
Fang J, Islam W, Maeda H. Exploiting the dynamics of the EPR effect and strategies to improve the therapeutic effects of nanomedicines by using EPR effect enhancers. Adv Drug Deliv Rev 2020, 157: 142-160.
Farajpour Z, Rahbarizadeh F, Kazemi B, Ahmadvand D. A nanobody directed to a functional epitope on VEGF, as a novel strategy for cancer treatment. Biochemical and biophysical research communications 2014, 446(1): 132-136.
Farokhzad O C, Langer R. Impact of nanotechnology on drug delivery. Acs Nano 2009, 3(1): 16-20.
Finger, C., Escher, C. & Schneider, D. The single transmembrane domains of human receptor tyrosine kinases encode self-interactions. Sci Signal 2, ra56, doi:10.1126/scisignal.2000547 (2009).
Fowler P W, Helie J, Duncan A, Chavent M, Koldso H, Sansom MSP. Membrane stiffness is modified by integral membrane proteins. Soft Matter 12, 7792-7803, doi:10.1039/c6sm01186a (2016).
Geisler, H. C., et al. EGFR-targeted ionizable lipid nanoparticles enhance in vivo mRNA delivery to the placenta. J Control Release 371, 455-469 (2024).
Genheden S, Ryde U. The M M/PBSA and M M/GBSA methods to estimate ligand-binding affinities. Expert opin Drug Discov 2015, 10(5): 449-461.
Goddard T D, Huang C C, Meng E C, Petersen E F, Couch G S, Morris J H, et al. UCSF ChimeraX: Meeting modern challenges in visualization and analysis. Protein Sci 2018, 27 (1): 14-25.
Goldman A J, Cox R G, Brenner H. Slow Viscous Motion of a Sphere Parallel to a Plane Wall 0.2. Couette Flow. Chem Eng Sci 1967, 22(4): 653-&.
Golfetto, O., Hinde, E. & Gratton, E. Laurdan fluorescence lifetime discriminates cholesterol content from changes in fluidity in living cell membranes. Biophys J 104, 1238-1247, doi:10.1016/j.bpj.2012.12.057 (2013).
Grillo I, Morfin I, Prevost S. Structural Characterization of Pluronic Micelles Swollen with Perfume Molecules. Langmuir 2018, 34(44): 13395-13408.
Hama S, Sakai M, Itakura S, Majima E, Kogure K. Rapid modification of antibodies on the surface of liposomes composed of high-affinity protein A-conjugated phospholipid for selective drug delivery. Biochem Biophys Rep 2021, 27: 101067. doi:10.1016/j.bbrep.2021.101067 (2021).
Haque M E, McIntosh T J, Lentz B R. Influence of lipid composition on physical properties and PEG mediated fusion of curved and uncurved model membrane vesicles: “Nature's own” fusogenic lipid bilayer. Biochemistry 2001, 40(14): 4340-4348.
Heldin C H, Rubin K, Pietras K, Ostman A. High interstitial fluid pressure—an obstacle in cancer therapy. Nat Rev Cancer 2004, 4(10): 806-813.
Hessa T, Meindl-Beinker N M, Bernsel A, Kim H, Sato Y, Lerch-Bader M, et al. Molecular code for transmembrane-helix recognition by the Sec61 translocon. Nature 2007, 450(7172): 1026-1030, doi:10.1038/nature06387 (2007).
Hmila I, Saerens D, Ben Abderrazek R, Vincke C, Abidi N, Benlasfar Z, et al. A bispecific nanobody to provide full protection against lethal scorpion envenoming. FASEB journal: official publication of the Federation of American Societies for Experimental Biology 2010, 24(9): 3479-3489.
Hubert P, Sawma P, Duneau J P, Khao J, Henin J, Bagnard D, et al. Single-spanning transmembrane domains in cell growth and cell-cell interactions More than meets the eye? Cell Adhes Migr 2010, 4(2): 313-324.
Ingolfsson H I, Melo M N, van Eerden F J, Arnarez C, Lopez C A, Wassenaar T A, et al. Lipid organization of the plasma membrane. J Am Chem Soc 2014, 136(41): 14554-14559, doi:10.1021/ja507832e (2014).
Ishida T, Kirchmeier M J, Moase E H, Zalipsky S, Allen T M. Targeted delivery and triggered release of liposomal doxorubicin enhances cytotoxicity against human B lymphoma cells. Bba-Biomembranes 2001, 1515(2): 144-158.
Jeon S, Jun E, Chang H, Yhee J Y, Koh E Y, Kim Y, et al. Prediction the clinical EPR effect of nanoparticles in patient-derived xenograft models. J Control Release 2022, 351: 37-49, doi:10.1016/j.jconrel.2022.09.007 (2022).
Jorgensen W L, Chandrasekhar J, Madura J D, Impey R W, Klein M L. Comparison of Simple Potential Functions for Simulating Liquid Water. J Chem Phys 1983, 79(2): 926-935.
Jovcevska I, Zupanec N, Kocevar N, Cesselli D, Podergajs N, Stokin C L, et al. TRIM28 and beta-actin identified via nanobody-based reverse proteomics approach as possible human glioblastoma biomarkers. Plos one 2014, 9(11): e113688.
Kaiser H J, Orlowski A, Rog T, Nyholm T K M, Chai W G, Feizi T, et al. Lateral sorting in model membranes by cholesterol-mediated hydrophobic matching. P Nat/ Acad Sci USA 2011, 108(40): 16628-16633.
Kelley, L. A., Mezulis, S., Yates, C. M., Wass, M. N. & Sternberg, M. J. The Phyre2 web portal for protein modeling, prediction and analysis. Nat Protoc 10, 845-858, doi:10.1038/nprot.2015.053 (2015).
Khaleghi S, Rahbarizadeh F, Ahmadvand D, Hosseini H R M. Anti-HER2 VHH Targeted Magnetoliposome for Intelligent Magnetic Resonance Imaging of Breast Cancer Cells. Cell Mol Bioeng 2017, 10(3): 263-272.
Kim E-M, Jeong H-J. Liposomes: Biomedical Applications. Chonnam medical journal 2021, 57(1): 27.
Kollmer M, Close W, Funk L, Rasmussen J, Bsoul A, Schierhorn A, et al. Cryo-EM structure and polymorphism of Aβ amyloid fibrils purified from Alzheimer's brain tissue. Nature Communications 2019, 10(1): 4760.
Kozlowska D, Biswas S, Fox E K, wu B, Bolster F, Edupuganti op, et al. Gadolinium-loaded polychelating amphiphilic polymer as an enhanced MRI contrast agent for human multiple myeloma and non Hodgkin's lymphoma (human Burkitt's lymphoma). Rsc Adv 2014, 4(35): 18007-18016, doi:10.1039/c3ra45400b (2014).
Lace I, Cotroneo E R, Hesselbarth N, Simeth N A. Artificial peptides to induce membrane denaturation and disruption and modulate membrane composition and fusion. Journal of Peptide Science 2023, 29(5): e3466.
Leserman, L. D., Machy, P. & Barbet, J. Cell-specific drug transfer from liposomes bearing monoclonal antibodies. Nature 293, 226-228, doi:10.1038/293226a0 (1981).
Li J, Wang X L, Zhang T, Wang C L, Huang Z J, Luo X, et al. A review on phospholipids and their main applications in drug delivery systems. Asian J Pharm Sci 2015, 10(2): 81-98, doi:10.1016/j.ajps.2014.09.004 (2015).
Li L, Xu S, Yan H, Li X, Yazd H S, Li X, et al. Nucleic Acid Aptamers for Molecular Diagnostics and Therapeutics: Advances and Perspectives. Angew Chem Int Ed Engl 2021, 60(5): 2221-2231.
Li N, Weng D, Wang S M, Zhang Y, Chen S S, Yin Z F, et al. Surfactant protein-A nanobody-conjugated liposomes loaded with methylprednisolone increase lung-targeting specificity and therapeutic effect for acute lung injury. Drug Deliv 2017, 24(1): 1770-1781.
Liang X M, Mao G Z, Ng K Y S. Mechanical properties and stability measurement of cholesterol-containing liposome on mica by atomic force microscopy. J Colloid Interf Sci 2004, 278(1): 53-62, doi:10.1016/j.jcis.2004.05.042 (2004).
Lin W S, Malmstadt N. Liposome production and concurrent loading of drug simulants by microfluidic hydrodynamic focusing. Eur Biophys J 2019, 48(6): 549-558.
Liu, Manman, Li Li, Duo Jin, and Yangzhong Liu. “Nanobody-A versatile tool for cancer diagnosis and therapeutics.” Wiley Interdisciplinary Reviews: Nanomedicine and Nanobiotechnology 13, no. 4 (2021): e1697.
Liu Y, Castro Bravo K M, Liu J. Targeted liposomal drug delivery: a nanoscience and biophysical perspective. Nanoscale Horiz 2021, 6(2): 78-94, doi:10.1039/d0nh00605j (2021).
Liu Y N, Scrivano L, Peterson J D, Fens M H A M, Hernandez 1B, Mesquita B, et al. EGFR-Targeted Nanobody Functionalized Polymeric Micelles Loaded with mTHPC for Selective Photodynamic Therapy. Mol Pharmaceut 2020, 17(4): 1276-1292, doi:10.1021/acs.molpharmaceut.9b01280 (2020).
Lomize A L, Lomize M A, Krolicki S R, Pogozheva I D. Membranome: a database for proteome-wide analysis of single-pass membrane proteins. Nucleic Acids Research 2017, 45(D1): D250-D255, doi:10.1093/nar/gkw712 (2017).
Lujan H, Griffin W C, Taube J H, Sayes C M. Synthesis and characterization of nanometer-sized liposomes for encapsulation and microRNA transfer to breast cancer cells. Int J Nanomedicine 2019, 14: 5159-5173.
Lygina A S, Meyenberg K, Jahn R, Diederichsen U. Transmembrane Domain Peptide/Peptide Nucleic Acid Hybrid as a Model of a SNARE Protein in Vesicle Fusion. Angew Chem Int Edit 2011, 50(37): 8597-8601.
Ma H, Liu J, Ali M M, Mahmood M A, Labanieh L, Lu M, et al. Nucleic acid aptamers in cancer research, diagnosis and therapy. Chem Soc Rev 2015, 44(5): 1240-1256, doi:10.1039/c4cs00357h (2015).
Majumder A, Sandhu M, Banerji D, Steri V, Olshen A, Moasser M M. The role of HER2 and HER3 in HER2-amplified cancers beyond breast cancers. Sci Rep 2021, 11(1): 9091, doi:10.1038/s41598-021-88683-w (2021).
Mamot C, Drummond D C, Noble C O, Kallab V, Guo Z X, Hong K L, et al. Epidermal growth factor receptor-targeted immunoliposomes significantly enhance the efficacy of multiple anticancer drugs in vivo. Cancer Research 2005, 65(24): 1 1631-11638.
Marsh D. Thermodynamics of phospholipid self-assembly. Biophys J 2012, 102(5): 1079-1087, doi:10.1016/j.bpj.2012.01.049 (2012).
McQuarrie D A, Jachimowski C, Russell M. Kinetics of small systems. II. The Journal of Chemical Physics 1964, 40(10): 2914-2921.
Menon, I., Zaroudi, M., Zhang, Y. Z., Aisenbrey, E. & Hui, L. W. Fabrication of active targeting lipid nanoparticles: Challenges and perspectives. Mater Today Adv 16(2022).
Mesquita B S, Fens M, Di Maggio A, Bosman EDC, Hennink W E, Heger M, et al. The Impact of Nanobody Density on the Targeting Efficiency of PEGylated Liposomes. Int J Mol Sci 2022, 420 23(23).
Mesquita, Bárbara, Michal Heger, and Sabrina Oliveira. “Enhanced phototoxicity and specificity of A1PCS4 when loaded in liposomes conjugated with a MET-targeting nanobody.” Photodiagnosis and Photodynamic Therapy 46 (2024): 104123.
Miao, L., Zhang, Y. & Huang, L. mRNA vaccine for cancer immunotherapy. Mol Cancer 20, 41 (2021).
Mitchell M J, Billingsley M M, Haley R M, Wechsler M E, Peppas N A, Langer R. Engineering precision nanoparticles for drug delivery. Nature Reviews Drug Discovery 2021, 20(2): 101-124, doi:10.1038/s41573-020-0090-8 (2021).
Mora N L, Findlay H E, Brooks N J, Purushothaman S, Ces 0, Booth P J. The membrane transporter lactose permease increases lipid bilayer bending rigidity. Biophys J 2021, 120(17): 3787-3794, doi:10.1016/j.bpj.2021.06.038 (2021).
Muyldermans S. Nanobodies: natural single-domain antibodies. Annu Rev Biochem 2013, 82: 775-797, doi:10.1146/annurev-biochem-063011-092449 (2013).
Nellis D F, Ekstrom D L, Kirpotin D B, Zhu J, Andersson R, Broadt T L, et al. Preclinical manufacture of an anti-HER2 scFv-PEG-DSPE, liposome-inserting conjugate. 1. Gram-scale production and purification. Biotechnol Prog 2005, 21(1) 205-220, doi:10.1021/bp049840y (2005).
Nguyen H, Maier J, Huang H, Perrone V, Simmerling C. Folding simulations for proteins with diverse topologies are accessible in days with a physics-based force field and implicit solvent. J Am 757 Chem soc 2014, 136(40): 13959-13962.
Nishimura T, Hirose S, Sasaki Y, Akiyoshi K. Substrate-Sorting Nanoreactors Based on Permeable Peptide Polymer Vesicles and Hybrid Liposomes with Synthetic Macromolecular Channels. Journal of the American Chemical Society 2020, 142(1): 154-161.
Oliveira S, Schiffelers R M, van der Veeken J, van der Meel R, Vongpromek R, van Bergen En Henegouwen P M, et al. Downregulation of EGFR by a novel multivalent nanobody-liposome platform. J Control Release 2010, 145(2): 165-175.
Panikar, Sandeep Surendra, Nehla Banu, Jesse Haramati, Susana del Toro-Arreola, Annie Riera Leal, and Pedro Salas. “Nanobodies as efficient drug-carriers: progress and trends in chemotherapy.” Journal of Controlled Release 334 (2021): 389-412.
Parchekani, J., Allahverdi, A., Taghdir, M. & Naderi-Manesh, H. Design and simulation of the liposomal model by using a coarse-grained molecular dynamics approach towards drug delivery goals. Sci Rep 12, 2371, doi:10.1038/s41598-022-06380-8 (2022).
Pardi, N., Hogan, M. J., Porter, F. W. & Weissman, D. mRNA vaccines—a new era in vaccinology. Nat Rev Drug Discov 17, 261-279 (2018).
Pardon E, Laeremans T, Triest S, Rasmussen S G, Wohlkonig A, Ruf A, et al. A general protocol for the generation of Nanobodies for structural biology. Nature protocols 2014, 9(3): 674-693.
Pattni B S, Chupin V V, Torchilin V P. New Developments in Liposomal Drug Delivery. Chem Rev 339 2015, 115(19): 10938-10966, doi:10.1021/acs.chemrev.5b00046 (2015).
Piper J W, Swerlick R A, Zhu C. Determining force dependence of two-dimensional receptorligand binding affinity by centrifugation. Biophys J 1998, 74(1): 492-513.
Rahbarizadeh, S, F, Ahmadvand D, Hosseini HRM. Anti-HER2 VI-IH Targeted Magnetoliposome for Intelligent Magnetic Resonance Imaging of Breast Cancer Cells. Cell Mol Bioeng 2017, 10(3): 263-272.
Rahman M M, Abosheasha M A, Ito Y, Ueda M. DNA-induced fusion between lipid domains of peptide-lipid hybrid vesicles. Chem Commun 2022, 58(84): 1 1799-1 1802.
Rahman M M, Ueda M, Hirose T, Ito Y. Spontaneous Formation of Gating Lipid Domain in Uniform-Size Peptide Vesicles for Controlled Release. J Am Chem soc 2018, 140(51): 17956-17961.
Rahman, M. M., et al. Chimeric nanobody-decorated liposomes by self-assembly. Nat Nanotechnol 19, 818-824 (2024).
Rigaud J L, Pitard B, Levy D. Reconstitution of membrane proteins into liposomes: application to energy-transducing membrane proteins. Biochim Biophys Acta 1995, 1231(3): 223-246.
Risselada H J, Kutzner C, Grubmuller H. Caught in the Act: Visualization of SNARE-Mediated Fusion Events in Molecular Detail (vol 12, pg 1049, 201 1). Chembiochem 2011, 12(9): cp27 cp27.
Ritzmann N, Thoma J, Hirschi S, Kalbermatter D, Fotiadis D, Muller D J. Fusion Domains Guide the Oriented Insertion of Light-Driven Proton Pumps into Liposomes. Biophys J 2017, 113(6): 1181-1186.
Rodriguez P L, Harada T, Christian D A, Pantano D A, Tsai R K, Discher D E. Minimal “Self” Peptides That Inhibit Phagocytic Clearance and Enhance Delivery of Nanoparticles. Science 2013, 339(6122): 971-975, doi:10.1126/science.1229568 (2013).
Romsicki Y, Sharom F J. Phospholipid flippase activity of the reconstituted P-glycoprotein multidrug transporter. Biochemistry 2001, 40(23): 6937-6947.
Roovers R C, Vosjan M J, Laeremans T, el Khoulati R, de Bruin R C, Ferguson K M, et al. A biparatopic anti-EGFR nanobody efficiently inhibits solid tumour growth. International journal of cancer Journal international du cancer 2011, 129(8): 2013-2024.
Roy A, Kucukural A, Zhang Y. I-Tasser: a unified platform for automated protein structure and function prediction. Nat Protoc 2010, 5(4): 725-738.
Sanjanwala, Dhruv, and Vandana Patravale. “Aptamers and nanobodies as alternatives to antibodies for ligand-targeted drug delivery in cancer.” Drug Discovery Today 28, no. 5 (2023): 103550.
Sayed-Tabatabaei, Leila, Jaleh Varshosaz, Shaghayegh Haghjooy Javanmard, and Sahel Soghrati. “Camouflaged liposomes by 11A4-nanobody for co-delivery of cisplatin and nitroxoline in breast cancer tumors: An in vitro/in vivo study.” Journal of Drug Delivery Science and Technology 71 (2022): 103273.
Schafer L V, de Jong D H, Holt A, Rzepiela A J, de Vries A H, Poolman B, et al. Lipid packing drives the segregation of transmembrane helices into disordered lipid domains in model membranes. P Nat/ Acad Sci USA 2011, 108(4): 1343-1348, doi:10.1073/pnas.1009362108 (2011).
Scheve C S, Gonzales P A, Momin N, Stachowiak J C. Steric Pressure between Membrane-Bound Proteins Opposes Lipid Phase Separation. Journal of the American Chemical Society 2013, 135(4): 1 185-1 188.
Sercombe L, Veerati T, Moheimani F, Wu S Y, Sood A K, Hua S. Advances and Challenges of Liposome Assisted Drug Delivery. Frontiers in Pharmacology 2015, 6, doi:ARTN 286 10.3389/fphar.2015.00286 (2015).
Settles E I, Loftus A F, McKeown A N, Parthasarathy R. The Vesicle Trafficking Protein Sar1 Lowers Lipid Membrane Rigidity. Biophys J 2010, 99(5): 1539-1545 doi:10.1016/j.bpj.2010.06.059 (2010).
Siegel, D. P. & Epand, R. M. The mechanism of lamellar-to-inverted hexagonal phase transitions in phosphatidylethanolamine: Implications for membrane fusion mechanisms. Biophys J 73, 3089-3111 (1997).
Takikawa M, Fujisawa M, Yoshino K, Takeoka S. Intracellular Distribution of Lipids and Encapsulated Model Drugs from Cationic Liposomes with Different Uptake Pathways. Int J Nanomedicine 2020, 15: 8401-8409.
Tarafdar P K, Chakraborty H, Dennison S M, Lentz B R. Phosphatidylserine Inhibits and Calcium Promotes Model Membrane Fusion. Biophys J 2012, 103(9): 1880-1889.
Tong J, Borbat P P, Freed J H, Shin Y K. A scissors mechanism for stimulation of SNARE-mediated lipid mixing by cholesterol. Proc Natl Acad Sci USA 2009, 106(13): 5141-5146.
Valdes-Tresanco M S, Valdes-Tresanco M E, Valiente P A, Moreno E. gmx_MMPBSA: A New Tool to Perform End-State Free Energy Calculations with GROMACS. J Chem Theory Comput 2021, 17(10): 6281-6291.
van der Meel R, Oliveira S, Altintas I, Haselberg R, van der Veeken J, Roovers R C, et al. Tumor targeted Nanobullets: Anti-EGFR nanobody-liposomes loaded with anti-IGF-I R kinase inhibitor for cancer treatment. J Control Release 2012, 159(2): 281-289.
Verhaar, Elisha R., Andrew W. Woodham, and Hidde L. Ploegh. “Nanobodies in cancer.” In Seminars in immunology, vol. 52, p. 101425. Academic Press, 2021.
Wan Y, Tan J, Asghar W, Kim Y T, Liu Y, Iqbal S M. Velocity effect on aptamer-based circulating tumor cell isolation in microfluidic devices. J Phys Chem B 2011, 115(47): 13891-13896, doi:10.1021/jp205511m (2011).
Wang B H, Zhang H F, An J Y, Zhang yw, Sun L L, Jin Y J, et al. Sequential Intercellular Delivery Nanosystem for Enhancing ROS-Induced Antitumor Therapy. Nano Lett 2019, 19(6): 3505-3518.
Wang C L, Liu Y L, Ji Z Y, Wang E J, Li R J, Jiang H, et al. Cruciforms: Assembling Single Crystal Micro- and Nanostructures from One to Three Dimensions and Their Applications in Organic Field-Effect Transistors. Chem Mater 2009, 21(13): 2840-2845.
Wang, Zhigang, Yufei Yan, Rui Li, Ying Zhu, and Gen Yang. “Artificially engineered vesicular vehicles for targeted therapy.” Extracellular Vesicle 4 (2024): 100047.
Woll S, Bachran C, Schiller S, Schroder M, Conrad L, Scherliess R, et al. Sortagging of liposomes with a murine CDI lb-specific VHH increases in vitro and in vivo targeting specificity of myeloid cells. EurJ Pharm Biopharm 2019, 134: 190-198.
Wu H, Yu M, Miao Y, He S, Dai Z, Song W, et al. Cholesterol-tuned liposomal membrane rigidity directs tumor penetration and anti-tumor effect. Acta Pharm Sin B 2019, 9(4): 858-870, doi:10.1016/j.apsb.2019.02.010 (2019).
Wu Y R, Sefah K, Liu H P, wang R W, Tan W H. DNA aptamer-micelle as an efficient detection/delivery vehicle toward cancer cells. P Nat/ Acad Sci USA 2010, 107(1) 5-10, doi:10.1073/pnas.0909611107 (2010).
Yang, Wenli, Yanyang Pang, Xi Wang, Zhiheng Lai, Yanda Lu, Shaojiang Zheng, and Wu Wang. “A novel CTLA-4 blocking strategy based on nanobody enhances the activity of dendritic cell vaccine-stimulated antitumor cytotoxic T lymphocytes.” Cell Death & Disease 14, no. 7 (2023): 406.
Yuen, Daniel, Orlagh M. Feeney, Leo Noi, Sudhir Shengule, Victoria M. McLeod, Pauline Reitano, Sammi Tsegay et al. “Nanobody-Mediated Cellular Uptake Maximizes the Potency of Polylysine Dendrimers While Preserving Solid Tumor Penetration.” ACS nano (2025).
Zhai, J., Fong, C., Tran, N. & Drummond, C. J. Non-Lamellar Lyotropic Liquid Crystalline Lipid Nanoparticles for the Next Generation of Nanomedicine. Acs Nano 13, 6178-6206 (2019).
Zhang, J., Xue, R., Ong, W. Y. & Chen, P. Roles of cholesterol in vesicle fusion and motion. Biophys J 97, 1371-1380 (2009).
Zhukovsky, M. A., Filograna, A., Luini, A., Corda, D. & Valente, C. Phosphatidic acid in membrane rearrangements. FEBS Lett 593, 2428-2451 (2019).
U.S. Pat. Nos. 4,925,661; 4,957,735; 5,599,831; 5,641,758; 5,786,214; 5,945,400; 6,087,171; 6,207,133; 6,214,388; 6,251,365; 6,268,174; 6,326,482; 6,458,593; 6,891,022; 7,479,276; 9,090,659; 10,639,277; 12,220,464; 20020002157; 20020012698; 20020025313; 20020197656; 20030044407; 20030105069; 20030232738; 20040022760; 20040115128; 20040115185; 20040132161; 20040219204; 20050148538; 20050152963; 20050158372; 20050186264; 20060019924; 20060228300; 20060228357; 20060269542; 20070065499; 20070086942; 20070134154; 20070134212; 20070231378; 20070248659; 20080241233; 20080299668; 20090053299; 20090087478; 20090169613; 20090175784; 20090196913; 20090220582; 20090269279; 20090324579; 20090324584; 20100047230; 20100047261; 20100129357; 20100166845; 20100173799; 20100209490; 20100239652; 20100255078; 20110097263; 20110150979; 20110229552; 20120021043; 20120231066; 20120258126; 20120269721; 20120288878; 20130224285; 20140023698; 20140186431; 20140288192; 20140314832; 20150147383; 20150343084; 20150366993; 20160045596; 20160067182; 20160145299; 20160250328; 20160303264; 20170022290; 20170173128; 20170226534; 20170232245; 20170258811; 20170258903; 20170355750; 20180009883; 20180021294; 20180086831; 20180135106; 20180318440; 20180319657; 20180353567; 20180363035; 20190040151; 20190062729; 20190071487; 20190110989; 20190322747; 20190330620; 20190351044; 20190351048; 20200024365; 20200069814; 20200215206; 20200216531; 20200276261; 20200276330; 20200318173; 20200392473; 20200407434; 20210030864; 20210079407; 20210100838; 20210169896; 20210222165; 20210275588; 20210277140; 20210292400; 20210308277; 20210317187; 20220002433; 20220054640; 20220064226; 20220091110; 20220195006; 20220249594; 20220257786; 20220280652; 20230010108; 20230055473; 20230057350; 20230115871; 20230136448; 20230165954; 20230174630; 20230241000; 20230241241; 20230241243; 20230272052; 20230303305; 20230321135; 20230340114; 20230355749; 20230390335; 20230390426; 20240002411; 20240018580; 20240024504; 20240026031; 20240033374; 20240044770; 20240082356; 20240173426; 20240182561; 20240201167; 20240238201; 20240263161; 20240299553; 20240342195; 20240424147; and 20250011848;

### SUMMARY OF THE INVENTION

The present technology provides a chemical modification-free biophysical approach to producing immunoliposomes in one step through the self-assembly of a chimeric nanobody (cNB) into liposome bilayers (FIG. 1A). cNB with over 98% purity was harvested from a cell factory, which consists of a nanobody (NB) head against epidermal growth factor receptor 2 (HER2), a flexible peptide linker, and a hydrophobic transmembrane peptide as an anchor. The lipids, drugs, and cNBs in an optimized ratio can self-assemble into HER2-targeting immunoliposomes. Therefore, this method allows mass production of immunoliposomes by simply supplying cNBs to the formula without significant adjustment of the existing manufacturing pipeline. Approximately 64% of therapeutic compounds can be encapsulated into 100 nm liposomes under simple stirring; meanwhile, up to 2,500 cNBs can be anchored on liposomal membranes without steric hindrance. Drug-loaded immunoliposomes increase cytotoxicity on HER2-overexpressing cancer cell lines by 10- to 20-fold, inhibit the growth of xenograft tumors by 3.4-fold, and improve survival by more than twofold. These immunoliposomes, which boost therapeutic efficacy, can be conveniently and efficiently produced without synthetic chemicals or chemical reactions and could pave the way for industrial production and clinical use.

The present technology bypasses undesired chemical conjugation but prepares drug-loaded immunoliposomes in one step (FIG. 1A). Briefly, chimeric nanobodies (cNB) were massively produced in bacteria, which consist of a nanobody (NB) against human epidermal growth factor receptor 2 (HER2), a flexible peptide linker, and a human single transmembrane domain (STMD). The lipids, drugs, and cNBs in an optimized ratio can self-assemble into HER2-targeting immunoliposomes (FIG. 1A).

The present technology provides immunotargeted liposomes in which the immune component is tethered to a liposome through a linker and a transmembrane peptide. The immune component, linker and transmembrane peptide are preferably a chimeric peptide, and the immune component is preferably a nanobody, resulting in a cNB. As an alternate, the hydrophobic transmembrane peptide in the cNB is replaced by a lipid acid, i.e., palmitic acid. This modification results in a more than 200-fold increase in cNB yield. Approximately 11.7 mg of cNB with a purity of 96% can be harvested from 40-ml supernatant. Similarly, a streamlined method is provided for producing epidermal growth factor receptor 2 (HER2)-targeting PEG-free mRNA-LNP through the self-assembly of cNBs, which bear palmitic acid as an anchor, along with lipids, and mRNA encoding the coronavirus spike protein (SP). During the preparation of mRNA-LNPs, over ~200 cNB were integrated to a ~70 nm LNP. The mRNA encapsulation efficiency was ~69%. The administration of these mRNA-LNP induced SP presentation on HER2-expressing tumor cell surfaces, triggering targeted immune killing (FIG. 2). In an animal study, compared to the negative control, tumor volumes in mice receiving cNB-decorated mRNA-LNPs decreased by 73.4%. In brief, the one-step manufacturing method for tumor-targeting mRNA-LNP could advance tumor immunotherapy.

The core of the present technology self-assembling iLP technology lies in the spontaneous interaction between transmembrane segment and lipids, which allows cNBs to be readily integrated into the lipid layer.

cNB have three domains: (1) a nanobody head against a specific target, e.g., epidermal growth factor receptor 2 (HER2), (2) a linker, e.g., a flexible peptide linker, and (3) a transmembrane domain, e.g., a hydrophobic peptide.

NB may be selected to target cancer-specific antigens, such as HER2, EGFR, and EpCAM. NB can repeatedly unfold and refold without significant aggregation or loss of specific binding. In contrast, full-length antibodies pose concerns with large size and immunogenicity. Complete antibody fragment and aptamer are sensitive to temperature, pH, and ionic strength. NB can be further humanized to improve biocompatibility.

Linker can be flexible (e.g., GGGGS SEQ ID NO: 007), semi-flexible (e.g., GGGGS-EAAAK SEQ ID NO: 023), or rigid (e.g., EAAAK SEQ ID NO: 009). The length of linker is typically larger than 1 nm, depending on the number of repeats of the basic unit. For example, a GGGGS SEQ ID NO: 007 linker with 8 repeats can extend to −14 nm in length.

Lipid formulations may be tuned for effective integration of cNBs into the lipid bilayer via the transmembrane segment and to achieve iLPs with optimal physicochemical properties. In brief, the integration of cNBs onto liposomal membranes not only formed a hydrophilic negatively charged protein sheath but also altered the biophysical properties of iLPs, including membrane fluidity, thermostability, and rigidity. Optimizing rigidity and stability of the iLPs is important, as both significantly impact liposomal drug performance. The enhanced rigidity improves colloidal stability under fluid shear stress, minimizes drug leakage, and promotes tissue penetration. Optimal stability ensures regulatory compliance, industrial manufacturability and distribution, and treatment effectiveness. Rigidity and stability of iLPs can be optimized by adjusting lipid composition and the number of surface-decorated cNBs.

DMPC has excellent miscibility and interaction with α-helical peptide, leading to the formation of highly stable iLP. DOPE bestows iLPs with the capability to engage in membrane fusion with target cells, facilitating the cytosolic drug delivery. Chol content may be reduced to 10%, as higher Chol levels lead to highly ordered lipid phase and segregation of membrane proteins. α-helical peptide may prefer lipid with low melting temperature (Tm). High-Tm lipid may impede the integration of α-helical peptide into the membrane and compromise the stability of decorated cNBs. Even if α-helical peptides do efficiently integrate into the membranes primarily composed of high-Tm lipid, it could yield a more rigid and well-structured membrane. While highly stiff iLPs have high stability, they may encounter challenges in tissue penetration, have a limited capacity for encapsulating various drugs, and could even provoke immune responses, thereby impacting drug delivery.

DPPC (saturated, double chain, C16) is a primary phospholipid in mammalian membranes. It forms a stable bilayer at 41° C., with membrane rigidity and fluidity tunable via Chol. DPPC and Chol can be combined to form unilamellar vesicles with well-controlled sizes, ideal for encapsulating various drugs. cNBs, possessing a palmitic tail like DPPC, can readily integrate into DPPC/Chol LPs, ensuring homogeneous distribution across the membranes without causing phase segregation or cNB-rich microdomain formation.

When 1,300 cNBs anchor onto liposomal membranes (the interval between two nearly cNBs is ~7 nm), the NB-peptide spacer domains protruding from the membranes spontaneously create a hydrophilic, negatively charged protein sheath. This property is achieved without PEG.

The transmembrane segment can insert into a lipid membrane wherein the linker and nanobody head are external to the liposome, and the liposome contains cargos.

Various payloads, such as therapeutic acids, proteins, peptides, chemical compounds, can be loaded into iLPs. For example, iLPs can be used as mRNA nanocarriers for targeted delivery, aiding in situ CAR-T cell production; iLPs can also carry CRISPR/Cas9 for more precise gene editing. Beyond cancer therapy, iLPs could be beneficial in treating infections, pain, and other diseases.

In some embodiments, the hydrophobic peptide as an anchor may be substituted with a lipid acid, which can result in ~200-fold increase in cNB production (e.g., anti-HER2 NB-(GGGGS SEQ ID NO: 007)₈-palmitic tail vs anti-HER2 NB-(GGGGS SEQ ID NO: 007)₈-hydrophobic peptide).

The lipid tail may be a palmitic acid tail (saturated, single chain, C16). Integrating cNBs with a palmitic tail into lipid layer could make the prepared iLPs or LNPs resemble natural vesicles.

Eukaryotic systems offer several types of lipidation, including palmitoylation, myristoylation, prenylation, acylation, and glypiation. A myristic tail (saturated, single chain, C14), an octanoyl tail (saturated, single chain, C8), and other lipid acids as anchors can also be used for preparation of cNBs.

Leveraging pre-existing immunity generated from common vaccines, such as the hepatitis B vaccine, tumor-targeting mRNA-LNP could be used to eliminate tumor cells expressing relevant pathogen antigens.

Two humanized NBs targeting EGFR and EpCAM with EC₅₀ of 0.23 nM and 0.96 nM in ELISA, respectively significantly reduced cytokine release in vitro, including IFN-7, IL-10, IL-6, and TNF-α.

cNB can be flexibly customized. The cNB comprises a nanobody, a peptide linker, and a transmembrane segment.

In the cNB, the nanobody portion can be customized based on specific requirements. For instance, the nanobody head can be chosen to target HER2, EGFR, or EpCAM.

The nanobody may be an anti-HER2 Variable Heavy domain of Heavy chain (VHH) nanobody having the sequence SEQ ID NO: 002 ESGGGSVQSGGSLRLSCAASGYNFGWYCMGWFRQAPGKEREGVASIGGSSITKYSDSV KGRFTISRDNAKNTLYLQMNALKPEDAATYYCAARPEYDCDSLREAGWRYWGQGTQV TVSS.

The nanobody may be an anti-EGFR Variable Heavy domain of Heavy chain (VHH) nanobody having the sequence SEQ ID NO: 003 EVQLVESGGGSVQTGGSLRLTCAASGRTSRSYGMGWFRQAPGKEREFVSGISWRGDST GYADSVKGRFTISRDNAKNTVDLQMNSLKPEDTAIYYCAAAAGSAWYGTLYEYDYWG QGTQVTVSS.

The nanobody may be an anti-EpCAM Variable Heavy domain of Heavy chain (VHH) nanobody having the sequence SEQ ID NO: 004 QVQLVQSGGGSVQGGASLRLSCAASGGERNNYCVAWFRQAPGKEREVAAISRAASGA QTTTKYYVDSVKGRFTISQDTKNTATVYLQMNSHKPEDTAYCTAKAKIYPPQCTGISRTI DYRGQGTQVTVSS.

The linker can be flexible (e.g., SEQ ID NO: 007 GGGGS), semi-flexible, and rigid (e.g., SEQ ID NO: 009 EAAAK) with varying lengths (e.g., with 8 repeats of basic unit). The peptide linker may have the sequence, which is related between 1 and 8 times. The engineered nanobody conjugate may further comprise a peptide affinity tag for purification purpose, e.g., SEQ ID NO: 006 HHHHHH.

The transmembrane segment can be peptide or lipid acids, such as a α-helical peptide and a palmitic acid.

The peptide transmembrane domain may comprise a peptide, e.g., HER2 STMD having the sequence SEQ. ID: 001 SIISAVVGILLVVVLGVVFGILI.

The fatty acylation signal peptide enables mammalian cells to modify palmitic acid or other fatty acids to the surface of the synthesized protein. The peptide fatty acylation signal is a palmitoylation signal having the sequence SEQ ID. NO: 005 MLCCMRRTKQ or a mysistolylation signal having the sequence SEQ ID NO: 008 MGSSKS. The cysteine residue may be palmitoylated.

The lipid acid tail of cNB can insert into a lipid layer, wherein linker and nanobody are external to the liposome or LNP, and the liposome contains cargos.

It is a further object to provide a method of making an immunoliposome composition, comprising: mixing the engineered nanobody according to claim 1 with a liposome composition; and forming 100 nm liposomes from the liposome composition, having between 200 to 2500 chimeric nanobodies per 100 nm liposome.

The engineered nanobody further may further comprise sequence SEQ ID NO: 006 HHHHHH, the peptide linker may comprise at least one sequence SEQ ID NO: 007 GGGGS, the nanobody may be one of an anti-HER2 nanobody, anti-EGFR nanobody, or an anti-EpCAM nanobody, and the peptide transmembrane domain or the peptide fatty acylation signal may comprise a single peptide transmembrane domain SEQ. ID: 001 SIISAVVGILLVVVLGVVFGILI.

Another object provides an engineered nanobody conjugate comprising: a peptide affinity tag, a Variable Heavy domain of Heavy chain (VHH) nanobody, a peptide linker, a cysteine residue, and a peptide fatty acylation signal. The VHH nanobody may be one of an anti-HER2 nanobody, anti-EGFR nanobody, or anti-EpCAM. The peptide affinity tag may have the sequence SEQ ID NO: 006 HHHHHH. The peptide linker may have the sequence (SEQ ID NO: 007 GGGGS)ₓ, and x is equal to or greater than 1 and equal to or less than 8. The peptide fatty acylation signal may be a palmitoylation signal. The palmitoylation signal may have the sequence SEQ ID NO: 005 MLCCMRRTKQ. The cysteine residue may be palmitoylated.

A further object provides an immunoliposome composition comprising chimeric nanobody (cNB).

A still further object provides an immunoliposome composition comprising the engineered nanobody conjugate.

The immunoliposome composition may increase cytotoxicity on HER2-overexpressing cancer cell lines by about 10 to about 20-fold, inhibit growth of xenograft tumors by about three to about four fold, and increase survival of a mammal in need thereof by at least two fold.

Another object provides a method of making an immunoliposome composition, said method comprising mixing a nanobody with a liposome composition. The nanobody may be an engineered nanobody conjugate.

It is also an object to provide an engineered nanobody, comprising an affinity tag (SEQ ID NO: 006 HHHHHH, the nano being selected from one of:

anti-HER2 SEQ ID NO: 002:
ESGGGSVQSGGSLRLSCAASGYNFGWYCMGWFRQAPGKEREGVASIGGSSITKYSDSV
KGRFTISRDNAKNTLYLQMNALKPEDAATYYCAARPEYDCDSLREAGWRYWGQGTQV
TVSS
anti-EGFR SEQ ID NO: 003
EVQLVESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYWG
QGTQVTVSS,
anti-EpCAM SEQ ID NO: 004
QVQLVQSGGGSVQGGASLRLSCAASGGERNNYCVAWFRQAPGKEREVAAISRAASGA
QTTTKYYVDSVKGRFTISQDTKNTATVYLQMNSHKPEDTAYCTAKAKIYPPQCTGISRTI
DYRGQGTQVTVSS,

a linker, e.g. SEQ ID NO: 007 GGGGS×1-10 (e.g., 2, 4, 6, 8 or 10), and
a membrane anchor, e.g., HER2 STMD SEQ ID NO: 001 SIISAVVGILLVVVLGVVFGILI.

The chimeric nanobody may comprise a cysteine conjugated through post-translational modifications, with palmitic acid, and a palmitoylation sequence SEQ ID NO: 005 MLCCMRRTKQ.

It is a further object to provide a myristoylated engineered nanobody, comprising a myristoylation sequence SEQ ID NO: 008 MGSSKS, an N-terminal myristoleic acid, a linker, which may comprise one or more SEQ ID NO: 007 GGGGS and one or more SEQ ID NO: 009 EAAAK, wherein the number of linker motifs is between 0 and 10, an affinity tag, e.g., SEQ ID NO: 006 HHHHHH, a nanobody, e.g.,

An Anti-HER2 nanobody SEQ ID NO: 002:
ESGGGSVQSGGSLRLSCAASGYNFGWYCMGWFRQAPGKEREGVASIGGSSITKYSDSV
KGRFTISRDNAKNTLYLQMNALKPEDAATYYCAARPEYDCDSLREAGWRYWGQGTQV
TVSS,
An Anti-EGFR nanobody SEQ ID NO: 003:
EVQLVESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYWG
QGTQVTVSS,
or
An Anti-EpCAM nanobody SEQ ID NO: 004:
QVQLVQSGGGSVQGGASLRLSCAASGGERNNYCVAWFRQAPGKEREVAAISRAASGA
QTTTKYYVDSVKGRFTISQDTKNTATVYLQMNSHKPEDTAYCTAKAKIYPPQCTGISRTI
DYRGQGTQVTVSS.

The linker may be (GGGGS SEQ ID NO: 007)₄, or (GGGGS SEQ ID NO: 007)₁(EAAAK SEQ ID NO: 009)₃, for example.

The chimeric nanobody may be an anti-HER2 NB with a linker and a hexahistidine tag,

STMD
SEQ ID NO: 010
HHHHHHESGGGSVQSGGSLRLSCAASGYNFGWYCMGWFRQAPGKEREGVAS
IGGSSITKYSDSVKGRFTISRDNAKNTLYLQMNALKPEDAATYYCAARPEYDCDSLREA
GWRYWGQGTQVTVSSGGGGSGGGGSGGGGSGGGGSGGGGSGGGGSGGGGSGGGGSS
IISAVVGILLVVVLGVVFGILI.

The chimeric nanobody may be an anti-HER2 NB with a linker and a hexahistidine tag and a palmitic tail SEQ ID NO: 011

HHHHHHESGGGSVQSGGSLRLSCAASGYNFGWYCMGWFRQAPGKEREGVAS IGGSSITKYSDSVKGRFTISRDNAKNTLYLQMNALKPEDAATYYCAARPEYDCDSLREA GWRYWGQGTQVTVSSGGGGSGGGGSGGGGSGGGGSGGGGSGGGGSGGGGSGGGGSC MLCCMRRTKQ.

The chimeric nanobody may be an Anti-EGFR NB with a hexahistidine tag, and a palmitic tail, and a linker (GGGGS SEQ ID NO: 007)₄ SEQ ID NO: 012

HHHHHHEVQLVESGGGSVQTGGSLRLTCAASGRTSRSYGMGWFRQAPGKERE FVSGISWRGDSTGYADSVKGRFTISRDNAKNTVDLQMNSLKPEDTAIYYCAAAAGSAW YGTLYEYDYWGQGTQVTVSSGGGGSGGGGSGGGGSGGGGSCMLCCMRRTKQ.

The chimeric nanobody may be an Anti-EGFR NB with a hexahistidine tag and a palmitic tail, and a linker (GGGGS SEQ ID NO: 007)₆ SEQ ID NO: 013

HHHHHHEVQLVESGGGSVQTGGSLRLTCAASGRTSRSYGMGWFRQAPGKERE FVSGISWRGDSTGYADSVKGRFTISRDNAKNTVDLQMNSLKPEDTAIYYCAAAAGSAW YGTLYEYDYWGQGTQVTVSSGGGGSGGGGSGGGGSGGGGSGGGGSGGGGSCMLCCM RRTKQ.

The chimeric nanobody may be an Anti-EGFR NB with a hexahistidine tag and a palmitic tail, and a linker (GGGGS SEQ ID NO: 007)₈ SEQ ID NO: 014

HHHHHHEVQLVESGGGSVQTGGSLRLTCAASGRTSRSYGMGWFRQAPGKERE FVSGISWRGDSTGYADSVKGRFTISRDNAKNTVDLQMNSLKPEDTAIYYCAAAAGSAW YGTLYEYDYWGQGTQVTVSSGGGGSGGGGSGGGGSGGGGSGGGGSGGGGSGGGGSG GGGSCMLCCMRRTKQ.

The chimeric nanobody may be an Anti-EpCAM NB with a hexahistidine tag, a palmitic tail, and a linker (GGGGS SEQ ID NO: 007)₄ SEQ ID NO: 015

HHHHHHQVQLVQSGGGSVQGGASLRLSCAASGGERNNYCVAWFRQAPGKER EVAAISRAASGAQTTTKYYVDSVKGRFTISQDTKNTATVYLQMNSHKPEDTAYCTAKA KIYPPQCTGISRTIDYRGQGTQVTVSSGGGGSGGGGSGGGGSGGGGSCMLCCMRRTKQ.

The chimeric nanobody may be an Anti-EpCAM NB with a hexahistidine tag, a palmitic tail, and a linker (GGGGS SEQ ID NO: 007)₆ SEQ ID NO: 016

HHHHHHQVQLVQSGGGSVQGGASLRLSCAASGGERNNYCVAWFRQAPGKER EVAAISRAASGAQTTTKYYVDSVKGRFTISQDTKNTATVYLQMNSHKPEDTAYCTAKA KIYPPQCTGISRTIDYRGQGTQVTVSSGGGGSGGGGSGGGGSGGGGSGGGGSGGGGSC MLCCMRRTKQ.

The chimeric nanobody may be an Anti-EpCAM NB with a palmitic tail, a hexahistidine tag, and a linker (GGGGS SEQ ID NO: 007)₈ SEQ ID NO: 017

HHHHHHQVQLVQSGGGSVQGGASLRLSCAASGGERNNYCVAWFRQAPGKER EVAAISRAASGAQTTTKYYVDSVKGRFTISQDTKNTATVYLQMNSHKPEDTAYCTAKA KIYPPQCTGISRTIDYRGQGTQVTVSSGGGGSGGGGSGGGGSGGGGSGGGGSGGGGSG GGGSGGGGSCMLCCMRRTKQ.

The chimeric nanobody may be an Anti-EGFR NB with a myristic tail, and (GGGGS SEQ ID NO: 007)₄ SEQ ID NO: 018 MGSSKSGGGGSGGGGSGGGGSGGGGSHHHHHHEVQLVESGGGSVQTGGSLRLTCAAS GRTSRSYGMGWFRQAPGKEREFVSGISWRGDSTGYADSVKGRFTISRDNAKNTVDLQM NSLKPEDTAIYYCAAAAGSAWYGTLYEYDYWGQGTQVTVSS.

The chimeric nanobody may be an Anti-EGFR NB with a myristic tail, and linker (GGGGS SEQ ID NO: 007)₁(EAAAK SEQ ID NO: 009)₃ SEQ ID NO: 019 MGSSKSGGGGSEAAAKEAAAKEAAAKHHHHHHEVQLVESGGGSVQTGGSLRLTCAAS GRTSRSYGMGWFRQAPGKEREFVSGISWRGDSTGYADSVKGRFTISRDNAKNTVDLQM NSLKPEDTAIYYCAAAAGSAWYGTLYEYDYWGQGTQVTVSS.

The chimeric nanobody may be an Anti-EpCAM NB with a myristic tail and linker (GGGGS SEQ ID NO: 007)₄ SEQ ID NO: 020 MGSSKSGGGGSGGGGSGGGGSGGGGSHHHHHHQVQLVQSGGGSVQGGASLRLSCAAS GGERNNYCVAWFRQAPGKEREVAAISRAASGAQTTTKYYVDSVKGRFTISQDTKNTAT VYLQMNSHKPEDTAYCTAKAKIYPPQCTGISRTIDYRGQGT QVTVSS.

The chimeric nanobody may be an Anti-EpCAM NB with a myristic tail (GGGGS SEQ ID NO: 007)₁(EAAAK SEQ ID NO: 009)₃ SEQ ID NO: 021 MGSSKSGGGGSEAAAKEAAAKEAAAKHHHHHHQVQLVQSGGGSVQGGASLRLSCAA SGGERNNYCVAWFRQAPGKEREVAAISRAASGAQTTTKYYVDSVKGRFTISQDTKNTA TVYLQMNSHKPEDTAYCTAKAKIYPPQCTGISRTIDYRGQGTQ VTVSS.

SEQ ID NO: 005 MLCCMRRTKQ can be replaced by other motifs that can induce palmitoylation, e.g., SEQ ID NO: 022 CMLCCMRRTKQ.

SEQ ID NO: 008 MGSSKS can be replaced by other motifs that can induce myristylation.

### BRIEF DESCRIPTION OF THE DRAWINGS

FIGS. 1A to 1B show a schematic of cNB and immunoliposome preparation.

FIG. 2 shows a schematic of HER2-targeting mRNA-LNPs. Palmitoylated nanobodies lipids, and mRNA encoding the coronavirus spike protein (SP) can spontaneously form HER2-targeting mRNA-LNPs. The administration of these mRNA-LNPs can induce SP expression and subsequently induce immune killing.

FIGS. 3A to 3H shows characterization of NB and engineered cNB.

FIGS. 4A to 4H show characterization of biophysical properties of cNB-LP.

FIGS. 5A to 5G show tumor treatment in vitro and in vivo.

FIGS. 6A-6F show characterization of NB

FIGS. 7A to 7C show simulation of NB and NB/HER2 complex with AlphaFold Multimer.

FIGS. 8A and 8B show simulation of NB and NB/HER2 complex with GROMACS.

FIGS. 9A and 9B show characterization of hydrogen bonds and electrostatic interactions between NB and HER2.

FIG. 10A shows a Gibbs free energy analysis of NB/HER2 complex with gmx_MMPBSA.

FIG. 10B shows an analysis of G_{gas} and Gₛₒₗᵥ of NB/HER2 complex.

FIGS. 10C and 10D show AA residues involved in NB/HER2 interaction and respective performance.

FIGS. 10E and 10F show a heatmap of these involved AA residues in the last 10 ns of molecular dynamics simulation.

FIGS. 11A to 11C shows a simulation of cNB structure with AlphaFold Multimer.

FIGS. 12A to 12D show characterization of produced cNB.

FIGS. 13A and 13B show electron microscopy of chimeric nanobody, plain liposomes, and chimeric nanobody decorated liposomes.

FIGS. 14A to 14F show a prediction of aggregates of chimeric nanobody.

FIGS. 15A to 15C show characterization with FRET assay and protein BCA assay.

FIGS. 16A to 16C show the permeability and thermostability of chimeric nanobody decorated liposomes.

FIGS. 17A to 17H show a computation of membrane stiffness of cNB-LP.

FIGS. 18A and 18B show atomic force microscopy measurements of plain liposomes and chimeric nanobody decorated liposomes.

FIGS. 19A and 19B show physical characteristics of chimeric nanobody decorated liposomes.

FIGS. 20A and 20B show interaction between chimeric nanobody decorated liposomes and two cancer cell lines.

FIGS. 21A to 21C show adhesion probability of chimeric nanobody decorated liposomes on HER2 overexpression cancer cells under different shear rates.

FIGS. 22A to 22G show the size of cNB-LP²⁰⁰⁰

FIGS. 23A to 23E show characterization of drug loading and release profiles.

FIGS. 24A to 24 C show characterization of treatment efficacy in vitro.

FIGS. 25A to 25E show tumor treatment with 5-Fluorouracil loaded chimeric nanobody decorated liposomes in vivo.

FIG. 26 shows Hematoxylin and Eosin staining of major organs and SK-BR-3 tumor in respective groups. Experiments were repeated five times (magnification 200×).

FIG. 27 shows NCI-87 tumor treatment with 5-Fluorouracil loaded chimeric nanobody decorated liposomes in vivo. NCI-N87 tumors obtained from each group at the end of treatment and respective tumor volume (1: PBS, 2: 5FU@cNB-LP, 3: 5FU@LP, and 4: 5FU; n=6, mean values shown; p<0.001, one-way ANOVA).

### DETAILED DESCRIPTION OF THE PREFERRED EMBODIMENTS

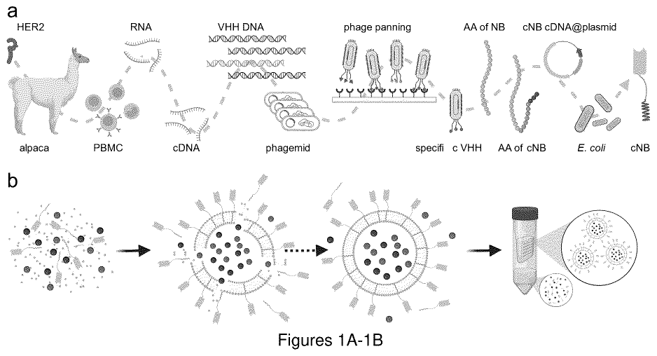

FIG. 1A shows a schematic Flowchart illustrating the manufacturing processes of cNB. Alpacas were immunized with the HER2 extracellular domain, and subsequent isolation of peripheral blood mononuclear cells (PBMCs) was performed. Total RNA was extracted from the PBMCs followed by cDNA synthesis for amplifying variable heavy domain of heavy chain (VHH) gene regions. The PCR products were then ligated into the phagemid vector, and E. coli cells were transformed with the ligated products and cultured. Colonies were recovered for the biopanning of phage displayed VHH libraries. One specific VHH was selected and sequenced to determine its amino acid sequences. Subsequently, in the design process amino acids encoding a hydrophilic linker (shown in yellow) and a STMD (shown in purple) were added to the C-terminus of the NB. The corresponding cDNA was synthesized and integrated into plasmids for the expression of the cNB using E. coli cells.

FIG. 1B shows self-assembly of lipoomes followed by purification.

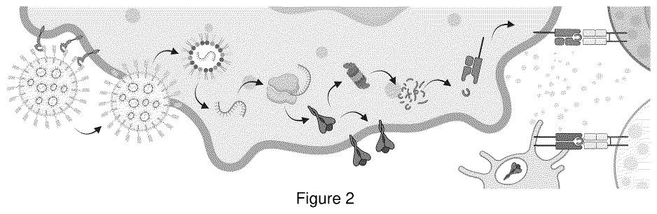

FIG. 2 shows a schematic of HER2-targeting mRNA-LNPs. Palmitoylated nanobodies lipids, and mRNA encoding the coronavirus spike protein (SP) can spontaneously form HER2-targeting mRNA-LNPs. The administration of these mRNA-LNPs can induce SP expression and subsequently induce immune killing.

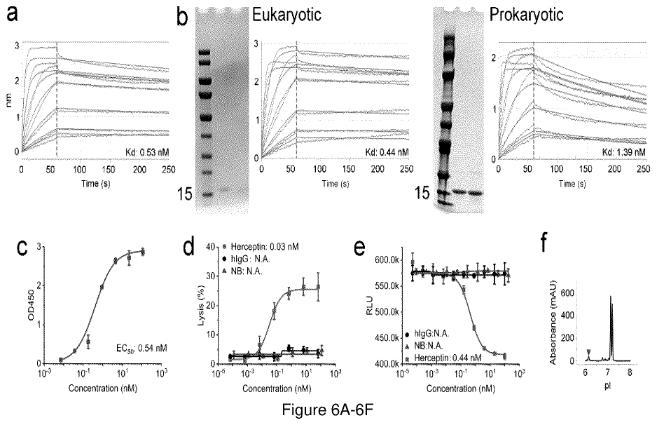

FIG. 6A shows a Kinetic analysis of NB produced by an alpaca.

FIG. 6B shows SDS-PAGE gel analysis and kinetic analysis of NBs produced with eukaryotic and prokaryotic systems, respectively.

FIG. 6C shows an EC₅₀ analysis of NB produced with prokaryotic systems (n=5).

FIG. 6D shows an ADCC analysis of NB produced with prokaryotic systems, Herceptin as a positive control, and hIgG as a negative control, respectively (n=5).

FIG. 6E shows inhibition of SK-BR-3 cell proliferation by NB produced with prokaryotic systems, Herceptin, and hIgG, respectively (n=5).

FIG. 6F shows isoelectric point analysis of NB produced with prokaryotic systems (n=3).

282 colonies were screened to identify 39 NBs specifically binding to HER2. One NB with Kd of 0.53 nM was selected for production, and E. coli was preferred over CHO cells for its cost-effective (FIGS. 6A-6B). The EC₅₀ of harvested NB was 0.54 nM (FIG. 6C). Antibody-dependent cellular cytotoxicity was not observed due to the absence of an Fc domain (FIG. 6D). The NB did inhibit HER2-overexpressing SK-BR-3 cells (FIG. 6E), indicating the NB solely has a targeting effect. Moreover, the NB is negatively charged at physiological pH (FIG. 6F). The NB/HER2 complex is stabilized by complementary structure, hydrogen bonds, and electrostatic interactions.

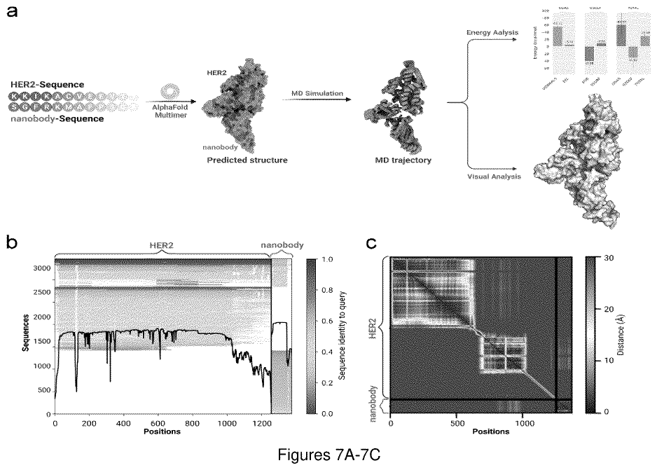

FIG. 7A simulation process with AlphaFold Multimer.

FIG. 7B shows the results of homology comparison between HER2 and NB. Larger value of sequences indicate higher simulation confidence.

FIG. 7C shows the distance matrix between amino acids derived from NB and HER2.

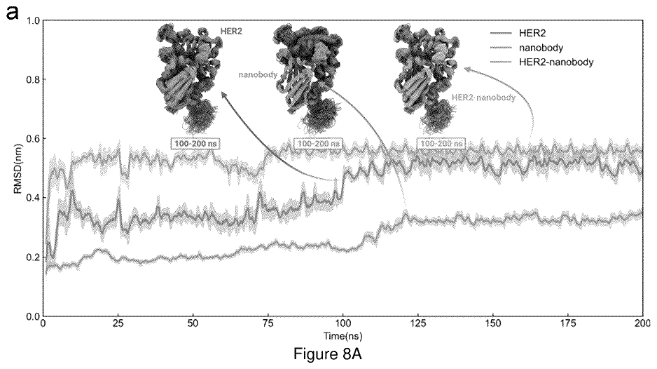

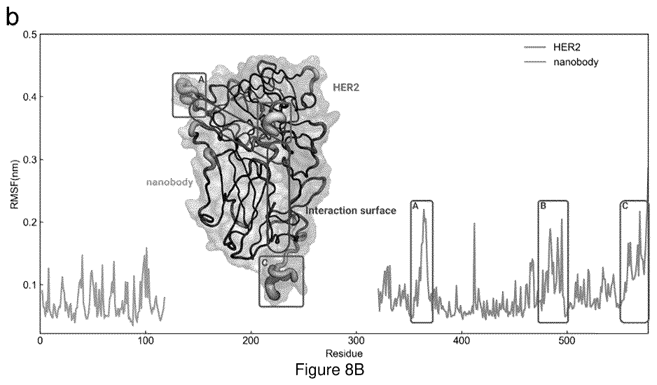

FIG. 8A shows an RMSD analysis of NB (pink), HER2 (blue), and NB/HER2 complex (green), respectively, throughout 200 ns of molecular dynamics simulation.

FIG. 8B shows an RMSF and B-factor analysis of NB (pink curve) and HER2 (blue curve) after 100 ns interaction.

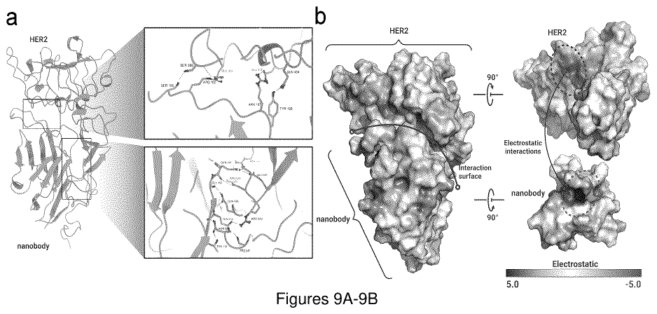

FIG. 9A shows a predicted hydrogen bonds involved in NB/HER2 complex between 100 ns and 200 ns of molecular dynamics simulation.

FIG. 9B shows a predicted electrostatic interactions between NB and HER2 between 100 ns and 200 ns of molecular dynamics simulation.

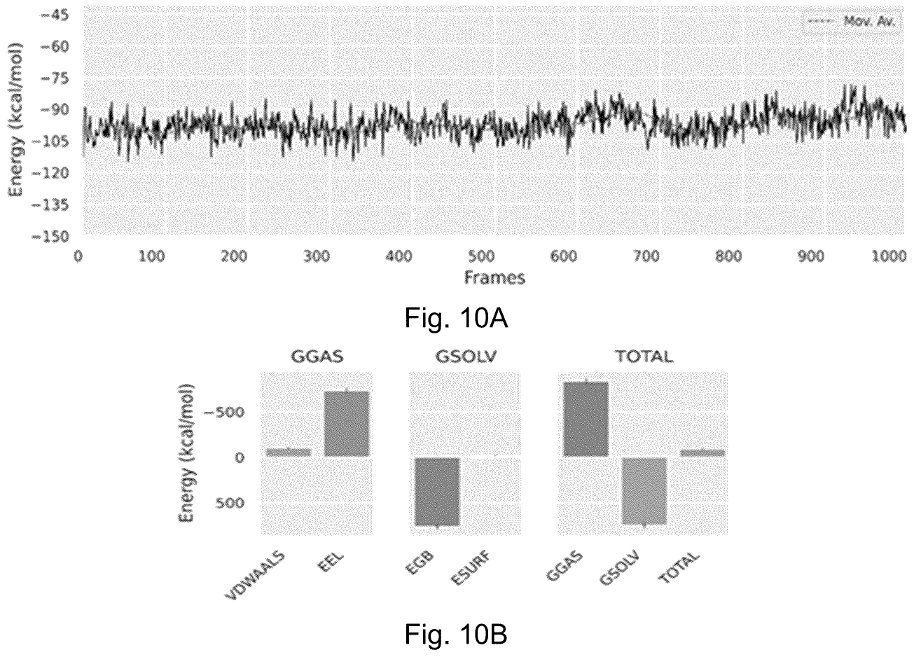

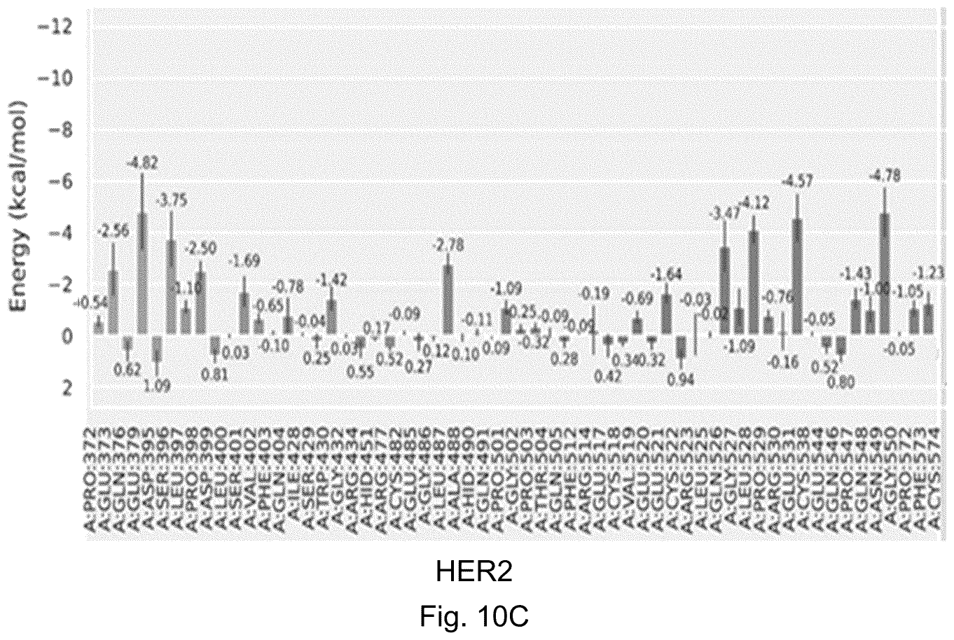

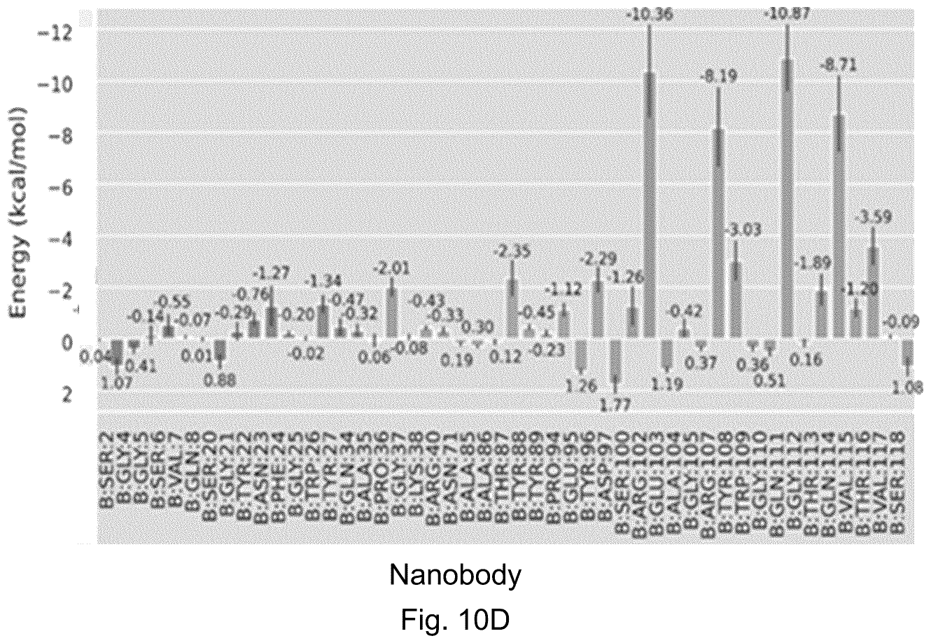

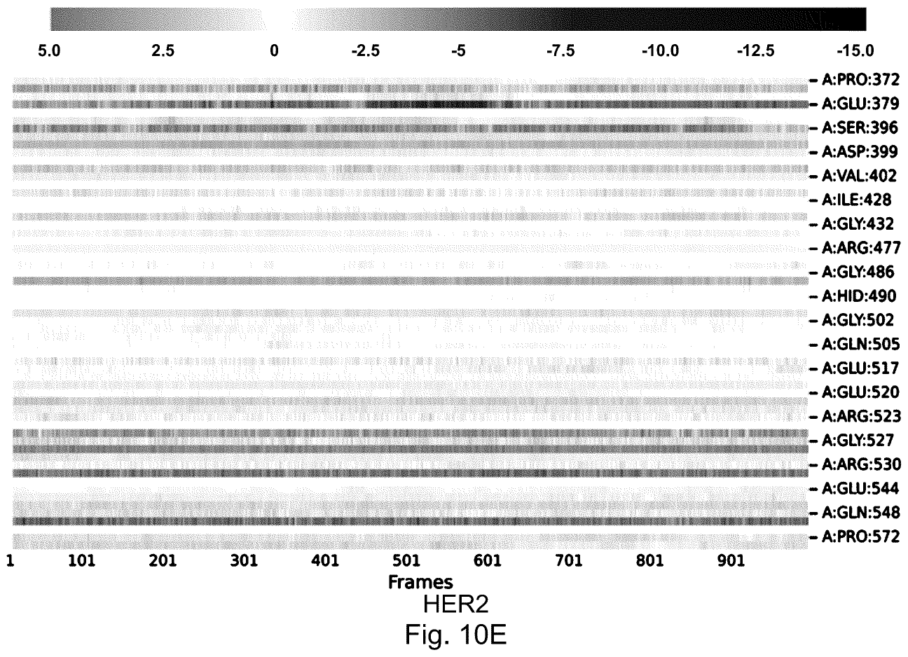

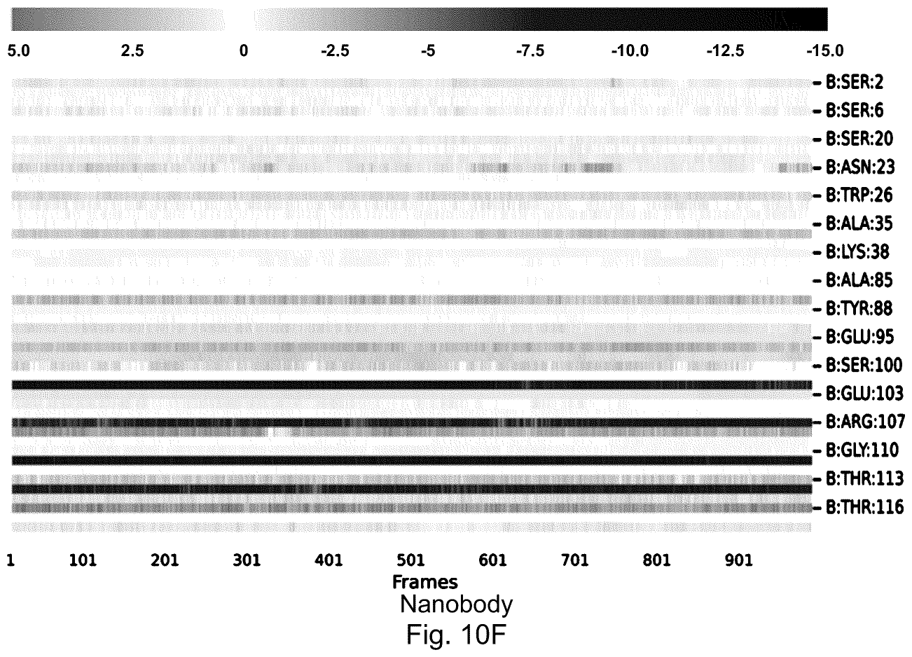

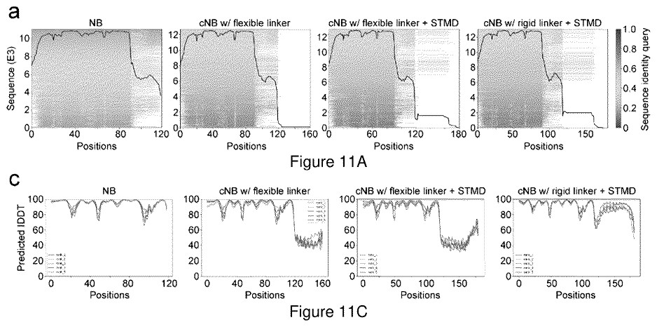

FIG. 10A shows the binding free energy of NB/HER2 complex in the last 10 ns of molecular dynamics simulation.

FIG. 10B shows an analysis of G_{gas} and Gₛₒₗᵥ of NB/HER2 complex.

FIG. 10C shows AA residues involved in HER2 interaction and respective performance.

FIG. 10D AA residues involved in Nanobody (NB) interaction and respective performance.

FIGS. 10E-10F show heatmaps of these involved AA residues in the last 10 ns of molecular dynamics simulation.

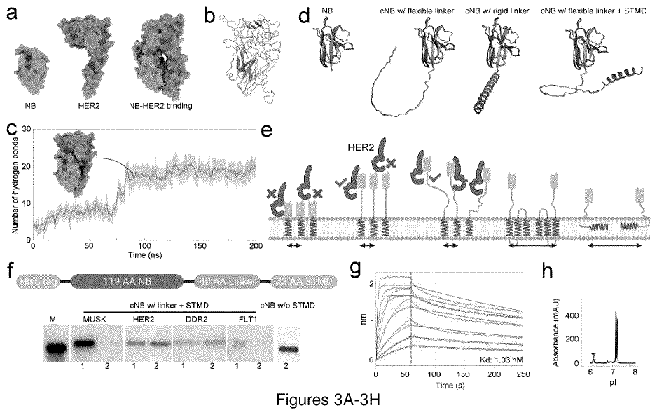

The NB primarily binds to domain IV and II of HER2 (FIG. 7). The complex achieved equilibrium, with the minimal root mean square fluctuation (RMSF) values being less than 0.1 nm (FIGS. 8A-8B). Only three regions displayed a relatively large RMSF of ~0.2 nm. These regions located outside the main interaction surface and thus would not weaken the overall equilibrium. The complex is stabilized by ~17 H-bonds and a complementary electrostatic potential surface between NB and HER2 (FIGS. 3A-3C, FIGS. 9A=9B). The average binding free energy of the NB/HER2 complex was−90.12 kcal/mol (FIG. 10A). The G_{gas} and Gₛₒₗᵥ were −838.05 and 747.94 kcal/mol, respectively (FIG. 10B). Several amino acid residuals of HER2 and NB primarily engage in the interaction (FIGS. 10C-10F). Beneficial mutations may further enhance the binding affinity.

FIG. 3A shows a structural model of identified anti-HER2 NB, HER2, and the NB/HER2 complex.

FIG. 3B shows crystal structure of NB/HER2 complex.

FIG. 3C shows prediction of formed hydrogen bonds in NB/HER2 complex throughout 200 ns of molecular dynamics simulation.

FIG. 3D shows a crystal structure of NB, cNB that harbors only a flexible linker, cNB that harbors only a rigid linker, and cNB that harbors a flexible linker and STMD.

FIG. 3E shows an illustration of potential steric hindrance effect and cNB grafting density influenced different linkers or transmembrane domains.

FIG. 3F shows a designed AA sequence of cNB (upper) and western blot of cNB with various STMD (lower) prepared at 15° C. for 16 h (1) and at 37° C. for 4 h (2).

FIG. 3G shows a kinetic analysis of cNB at pH 7.4. h, Isoelectric point analysis of cNB.

FIG. 3H shows isoelectric point analysis of cNB.

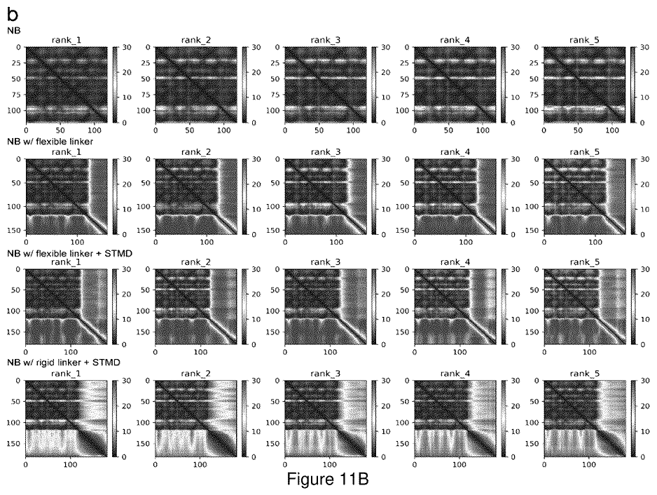

FIG. 11A shows the sequence coverage of NB, cNB with flexible linker, cNB with flexible linker and STMD, cNB with rigid linker and STMD.

FIG. 11B shows the predicted aligned error of each group.

FIG. 11C shows the predicted local distance difference test of each group.

The cNB was designed and its 3D structure predicted (FIG. 3D, FIGS. 11A-11C). A flexible (GGGGS SEQ ID NO: 007)₈ linker and a rigid (EAAAK SEQ ID NO: 009)₈ linker were investigated. The (GGGGS SEQ ID NO: 007)₈ fold-breaking linker enhances the mobility, reduces steric hindrance, and improves bioactivity of the NB. Meanwhile, the (GGGGS SEQ ID NO: 007)₈ linker maintains stability in aqueous solutions, which reduces the unfavorable interaction between the linker and the NB. In contrast, the (EAAAK SEQ ID NO: 009)₈ linker exhibits stiff structures, ensuring a fixed distance between the NB and the liposomal membranes. However, the limited movement of the NB may impact the NB/HER2 interaction (FIG. 3E). The hydrophobic (EAAAK SEQ ID NO: 009)₈ linker with an auto-cleavage feature may also lead to the loss of the NB, compromising targeted delivery. Multi-pass transmembrane proteins, monotopic membrane proteins, or β-sheet peptides are transmembrane proteins that occupy ample space, lowering their grafting density (FIG. 3E). Altogether, the cNB with a (GGGGS SEQ ID NO: 007)₈ linker of ~20 nm length was designed. The decoration of the cNBs on liposomal membranes could form a protein sheath layer with a thickness of at least ~6 nm. The hydrophilic and anionic sheath layer could prevent fouling and reduce phagocytic clearance.

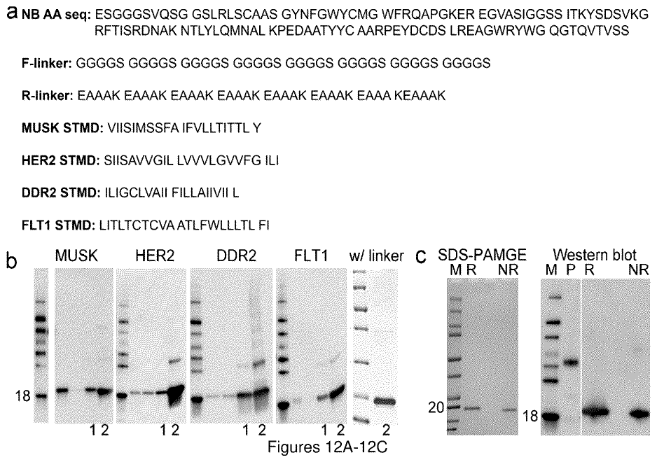

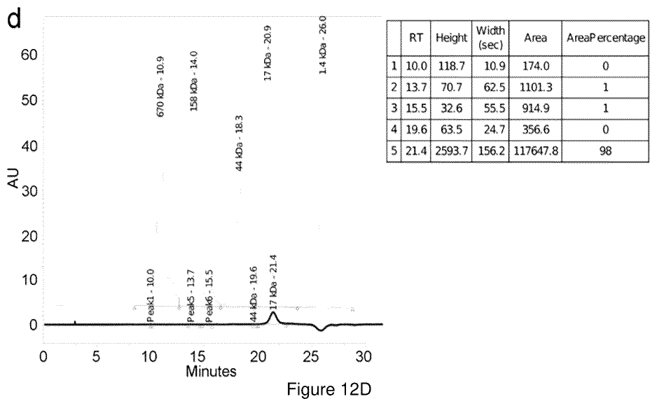

FIG. 12A shows an AA sequence of NB, flexible linker, rigid linker, and various STMD, respectively.

FIG. 12B shows a western blot of cNB with various STMD or without STMD prepared at 15° C. for 16 h (1) and at 37° C. for 4 h (2).

FIG. 12C shows SDS-PAMGE and western blot analysis of cNB harboring HER2 STMD (M: marker, P: positive control, R: reducing, NR: non-reducing.

FIG. 12D shows SEC-HPLC analysis of purified cNB and quantified purity.

A His6 tag was added to the N-terminus and a STMD to the C-terminus (FIG. 3F). Four human STMDs were compared (FIG. 12A). MUSK, HER2, and DDR2 are neutral, gradually increasing in hydrophobicity. MUSK and FLT1 have similar hydrophobicity, but FLT1 is negatively charged. Artificial peptides were excluded, known to induce membrane disruption.18 The produced cNBs can be harvested from the supernatant of cell lysate (FIG. 3F), with abundant cNBs found in the pellet (FIG. 12B). The expression level of four cNBs was similar, suggesting that the hydrophobicity and surface charge of STMD do not significantly influence cNB production. Indeed, STMD lowered the yield and solubility of cNB compared to the counterpart with only the (GGGGS SEQ ID NO: 007)₈ linker (FIG. 12B). Among the four variants, the HER2-harboring cNB showed a reliable yield, and thus we harvested this cNB with a purity of 98% for the subsequent studies (FIGS. 12C-12D). The Kd and the pI of cNB did not significant alter due to the adoption of the neural linker and STMD (FIGS. 3G-3H).

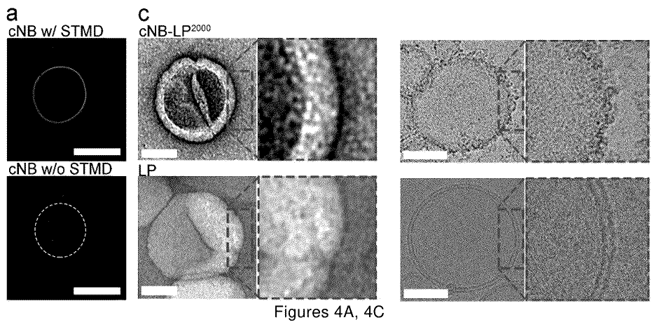

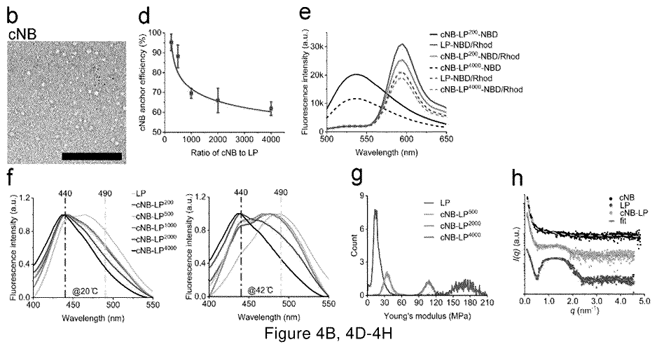

FIG. 4A shows immunofluorescence staining of a LP decorated with intact cNBs (upper) and STMD deficient cNBs (lower). Scale bar: 5 μm.

FIG. 4B shows a TEM image shows the morphology of cNBs. Scale bar: 100 nm.

FIG. 4C shows the cryo-TEM (left) and TEM (right) images show the membrane morphology of a typical cNB-LP²⁰⁰⁰ and a typical plain LP. Scale bar: 50 nm.

FIG. 4D shows decoration efficiency of cNB as a function of cNB quantity.

FIG. 4E shows fluorescence signals of FRET-pair labeled cNB-LPs and LPs.

FIG. 4F shows the fluorescence signals of Laurdan emission from various cNB-LP at 20° C. and 42° C.

FIG. 4G shows the stiffness distribution for free LPs and cNB-LPs (n=34-100).

FIG. 4H shows SAXS scattering curves measured for plain LP, free cNB, and cNB-LP²⁰⁰⁰ samples in solution.

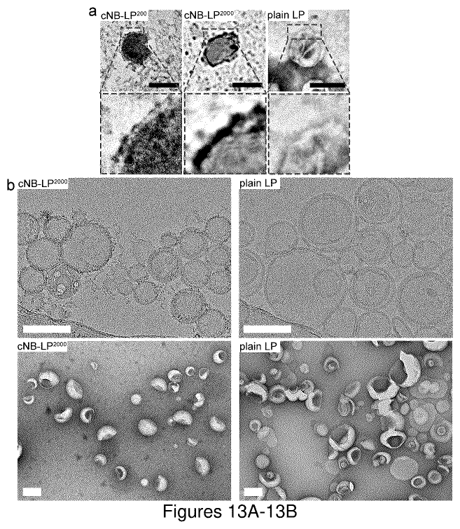

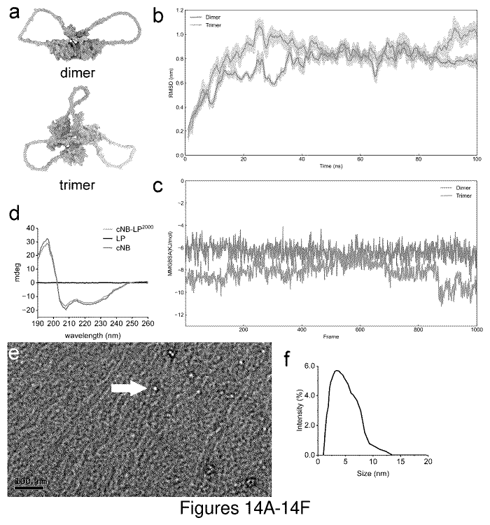

FIG. 14A shows a potential dimer and trimer of cNB.

FIG. 14B shows RMSD analysis of cNB dimer and trimer, respectively.

FIG. 14C shows MMGBSA analysis of cNB dimer and trimer, respectively (mean value±SD shown).

FIG. 14D shows CD spectra of plain LP, cNB, and cNB-LP²⁰⁰⁰ in PBS.

FIG. 14E shows a TEM image showing the morphology of cNBs or cNB aggregates indicated by the white arrow. Scale bar is 100 nm. Experiments were repeated three times.

FIG. 14F shows a size distribution of cNBs in PBS measured by dynamic light scattering.

cNB-decorated liposomes (cNB-LP) were prepared, and the anchored cNBs were observed with anti-His6 fluorescent antibodies (FIG. 4A). Transmission electron microscopy (TEM) visualized 100-nm plain LPs and cNB-LP prepared in two cNB-to-LP ratios, i.e., 200:1 (cNB-LP²⁰⁰) and 2000:1 (cNB-LP²⁰⁰⁰) (FIGS. 13A-13B). cNB clusters formed during sample dehydration and appeared as white dots with a diameter less than 8 nm (FIG. 4B). A coarse edge was observed in cNB-LPs as opposed to LPs (FIG. 4C). The membrane thickness of LPs was ~4 nm, which increased to −10 nm in cNB-LPs. Formation of cNB micelles was ruled out. Computational results showed that these cNBs were not arranged in a head-to-head and tail-to-tail manner (FIG. 14A). The low binding energy between cNBs revealed that the aggregation was highly unstable (FIGS. 14B-14C). The circular dichroism spectra of cNB and cNB-LP²⁰⁰⁰ exhibited the characteristic profile of an α-helix configuration (FIG. 14D), but not a robust helix-helix structure. The intensity ratio of 222/208 for cNB and cNB-LP²⁰⁰⁰ was only 0.80 and 0.85, respectively, indicating dispersed helical structures. Moreover, if cNB micelles were present, their diameter would likely measure ~25 nm or more, inconsistent with TEM and size analysis (FIGS. 14E-14F).

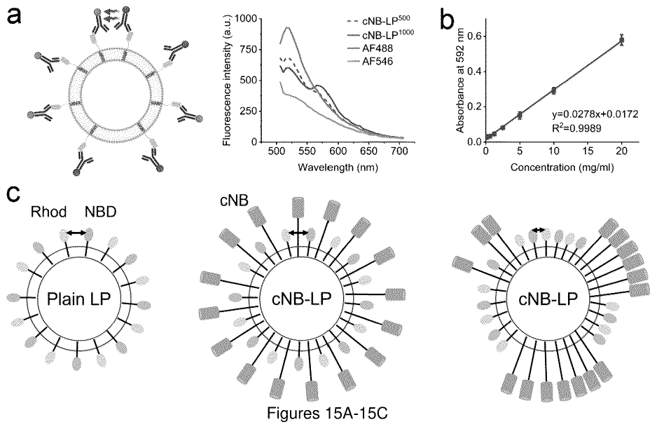

FIG. 15A shows a schematic of FRET analysis (left) and fluorescence signals of FRET emission of cNB decorated liposomes (right).

FIG. 15B shows an eight-point standard curve of BCA assay.

FIG. 15C shows FRET analysis of NBD-PE and RhodPE co-labeled plain LPs and cNB-LPs. The plain LP served as a negative control assuming that NBD and Rhod were homogeneously distributed across the membrane. If the presence of cNBs incorporated into the membranes as discrete blocks, the proximity between NBD and Rhod would be altered, and this change would be manifested in the signal intensity. In our experiments, we did not observe significant changes in signal intensity. Therefore, we eliminated this possibility and claimed that cNBs were homogeneous distributed across the membrane.

The fluorescence resonance energy transfer (FRET) assay also verified cNBs integration into liposomal membranes (FIG. 15A). Moreover, the fluorescence characteristics of cNB-LP¹⁰⁰⁰, compared to cNB-LP⁵⁰⁰, revealed more efficient energy transfer. The difference was attributed to increased cNB density and decreased inter-cNB distance on membranes. The anchor efficiency of cNB decreased from 95.3% to 61.9% when the mixing ratio of cNB-to-LP increased from 200 to 4,000 (FIG. 4D, FIG. 15B). Equivalently, ~190 to ~2,500 cNBs were integrated to 100-nm LPs. In contrast, a limited number of NBs can be grafted on LPs using conventional methods. One study reported the optimal number of NBs for achieving ideal targeting specificity and efficiency. The findings may not be applicable to our cNB-LPs due to the use of PEG and variations in liposomal size. Nevertheless, a few hundred cNBs are sufficient for targeted drug delivery, and our method can readily adjust the number of cNBs. We speculated that cNBs anchor onto both outer and inner membranes. The interval between two nearby cNBs in cNB-LP²⁰⁰ is ~10 nm if 190 cNBs fully incorporate into the outer membranes, and FRET occurs within this proximity. However, no fluorescence change was observed, indicating that adjacent cNBs were over 10 nm apart. A few cNBs might anchor on the inner membranes. Given the outer and inner surface areas of LPs are nearly identical, we presumed homogeneous anchoring of cNBs on two sides. Thus, for cNB-LP²⁰⁰, cNB-LP⁵⁰⁰, and cNB-LP¹⁰⁰⁰, ~95, ~220 and ~345 cNBs were on the outer membranes, resulting in intervals of ~18.2, ~9.3, and ~4.2 nm, respectively. The FRET data supported this calculation. We also deduced that cNBs were homogeneously distributed over the membranes rather than anchored as blocks (FIG. 4E, FIG. 15C), as evidenced by almost identical FRET efficiencies of cNB-LPs and LPs at 535 nm. The quantity of cNB did not impact its homogeneous distribution.

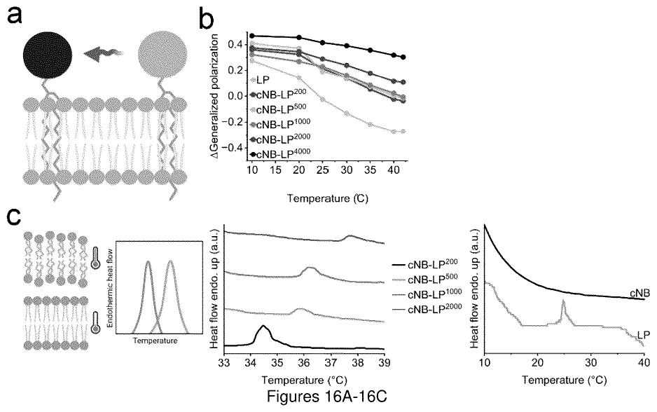

FIG. 16A shows the mechanism of Laurdan analysis. When membrane fluidity decreases, Laurdan spectrum blueshifts.

FIG. 16B shows averaged ΔGeneralized polarization values as a function of temperature in each group (n=5).

FIG. 16C shows differential scanning calorimetry analysis of different cNB-LP samples, LP, and cNB as a function of temperature (n=5).

Membrane fluidity was studied with a Laurdan assay (FIG. 4F, FIG. 16A). The fluid and gel phase lead to emission at 490 and 440 nm, respectively. At 20° C., cNB-LPs showed low membrane fluidity compared to the plain LPs. At 42° C., the emission peak of LPs was detected at 490 nm. The emissions of cNB-LP²⁰⁰, cNB-LP⁵⁰⁰, and cNB-LP¹⁰⁰⁰ shifted towards 490 nm. Only cNB-LP²⁰⁰⁰ and cNB-LP⁴⁰⁰⁰ kept the emission peak at 440 nm. In brief, the membrane fluidity of cNB-LPs and plain LPs increased with temperature (FIG. 16B). The larger the number of anchored cNBs, the lower the decline rate of polarization and the more stable the cNB-LPs. In addition, crystalline melting curves suggested that incorporating cNBs improved the thermostability of cNB-LPs (FIG. 16C). In controls, cNBs exhibited no noticeable phase transition, while the LPs displayed a melting temperature of −24° C. as expected.

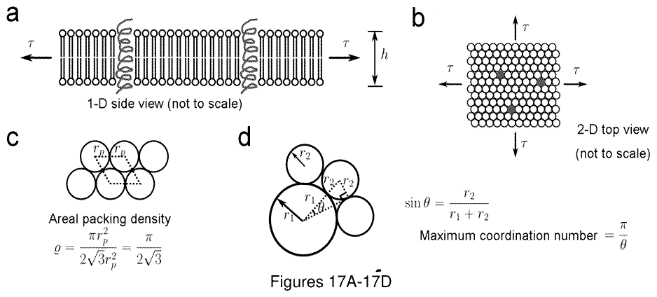

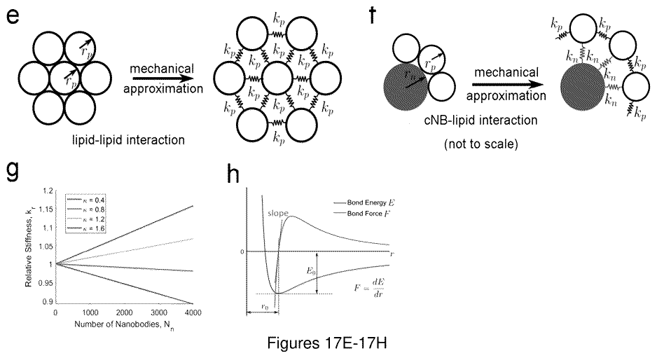

FIG. 17A shows a side view of a lipid bilayer with embedded STMD of cNB. The thickness of the lipid bilayer is h. The tilted angle of STMD in lipid bilayer was neglected. The membrane may be subjected to a tension r.

FIG. 17B shows an illustration of a 2D view of a lipid bilayer with embedded cNB. When a liposomal membrane is subjected to tension, this uniform tension can be represented by equal biaxial tension r.

FIG. 17C shows the areal packing density, ρ, of the lipids can be estimated by assuming rigid spheres of the lipid heads. The areal packing density from lipids was used to approximate the average packing density of the entire membrane.

FIG. 17D shows a calculation of the maximal coordination number, which is the maximum number of contacting molecules of radius r2 around a molecule of radius r1.

FIG. 17E shows an illustration of lipid-lipid interactions. rp is the radius of a lipid head and kp is the spring constant between lipids.

FIG. 17F shows an illustration of cNB-lipid interactions. rn is the radius of a NB and kn is the spring constant between a cNB and a lipid. Only three lipid heads were drawn for illustrative purposes.

FIG. 17G shows a predicted relative stiffness of the membrane as a function of the number of cNB for different ratios of kn/kp.

FIG. 17H shows schematics of inter-molecular interactions. The bonding force is the derivative of the bonding energy.

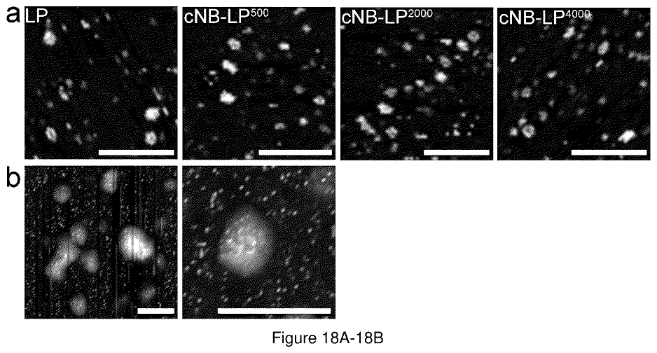

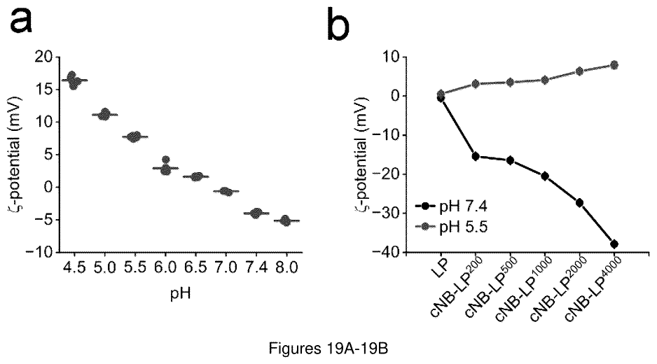

This was further examined with a computational model (FIGS. 17A-17F). cNBs can enhance the membrane stiffness when kₙ/kₚ>0.89, where kₙ is the interaction between an STMD and a lipid, and kₚ is the interaction between two adjacent lipids (FIG. 17G). The condition requires STMD-lipid interaction strength to be nearly equivalent to lipid-lipid interaction. Due to unknown value of kₙ and kₚ, kₙ/kₚ was calculated using the inter-molecular distance and bonding energy (FIG. 17H). The ΔGtransfer of STMD-lipid and lipid-lipid is−28.8 and −6 kcal/mol, respectively. Using G to approximate minimum bonding energy, we obtained E_{0,n}/E_{0,p}≈5. Therefore, more cNBs could increase the membrane stiffness (FIG. 17G). The finding was verified by measurements of rigidity in plain LPs and cNB-LPs. The Young's modulus values for plain LPs, cNB-LP⁵⁰⁰, cNB-LP²⁰⁰⁰, and cNB-LP⁴⁰⁰⁰ were 14.9, 34.4, 107.7, and 174.3 MPa, respectively (FIG. 4G, FIGS. 18A-18B). The stiffness distribution curves illustrated that as the number of cNBs increased, rigidity proportionally escalated.

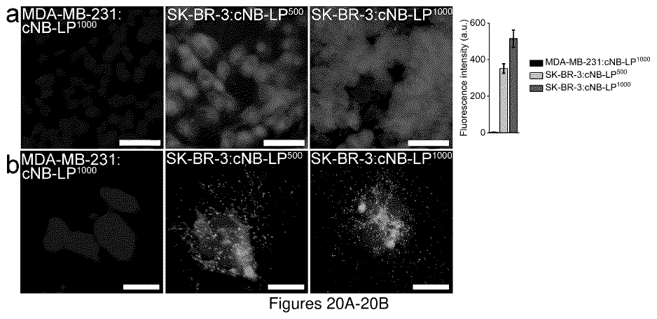

FIG. 19A shows the ζ-potential of cNB as a function of pH (n=5, mean values shown).

FIG. 19B shows the ζ-potential of various cNB-LP at pH 5.5 and pH 7.4 (n=15, mean value±SD).

The ζ-potential of free cNBs increased as the pH value decreased (FIG. 19A). The ζ-potential of cNB-LPs at pH 7.4 significantly decreased with increasing anchored cNBs. The opposite phenomenon was observed at pH 5.5 (FIG. 19B). In the acidic tumor microenvironment, positively charged cNB-LPs may facilitate the NB-HER2 binding.

FIG. 13A shows TEM images showing the morphology of cNB-LP²⁰⁰, and cNB-LP²⁰⁰⁰, and plain LPs. Scale bar: 100 nm. Experiments were repeated three times.

FIG. 13B shows cyroTEM and TEM images showing the morphology of multiple cNB-LP²⁰⁰⁰ and multiple plain LPs. Scale bar: 100 nm. Experiments were repeated three times.

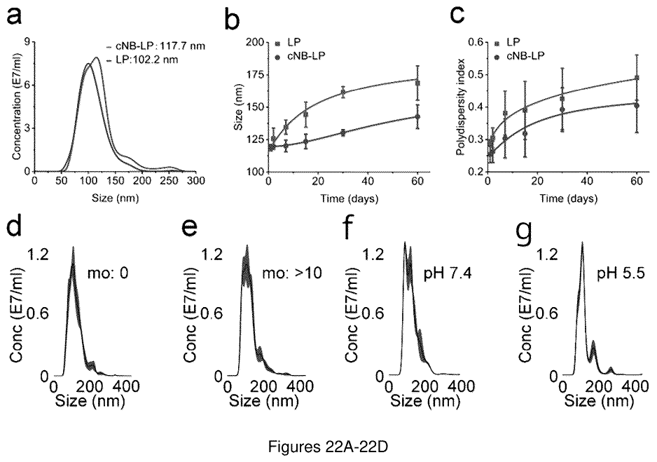

FIGS. 22A to 22E show the size of cNB-LP²⁰⁰⁰

FIG. 22A shows the size distribution of plain liposome and cNB-LP²⁰⁰⁰

FIG. 22B shows the respective size change of liposome and cNB-LP stored at −80° C. in PBS for up to 60 days Five biological replicates were measured at each time point (n=5, mean value±SD; p<0.05, t-test).

FIG. 22C shows the corresponding polydispersity index of liposome and cNB-LP. Five biological replicates were measured at each time point (n=5, mean value±SD; p>0.05, t-test).

FIG. 22D shows the theoretical model of cNB-LP adhesion with cancer cell surface under shear flow where the flow induced drag and torque is balanced by the adhesive force.

FIG. 22E shows changes in size of cNB-LP²⁰⁰⁰ over ten months (mean value±SD shown).

FIG. 22F shows changes in size of cNB-LP²⁰⁰⁰ at pH 7.4 for 30 h at 37° C. (mean value±SD shown).

FIG. 22G shows changes in size of cNB-LP²⁰⁰⁰ at pH 5.5 for 30 h at 37° C. (mean value±SD shown).

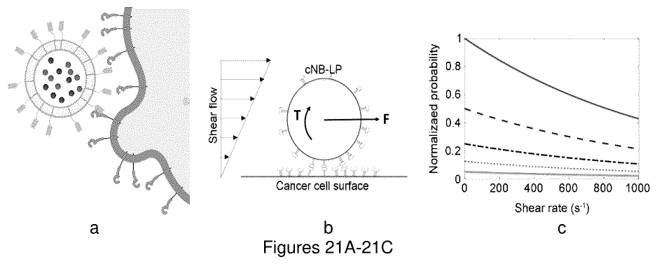

cNB-LPs can bind to native HER2 on cell membranes (FIGS. 20A-20B). A computational model revealed that more cNBs on membranes increase the adhesion probability of cNB-LPs under the same shear rate (FIGS. 21A-21C). In Small-Angle X-ray Scattering (SAXS), cNB-LP²⁰⁰⁰ exhibited a distinct change in patterns compared to LPs and cNBs, suggesting the integration of cNBs into the lipid membranes (FIG. 4H). SAXS detected an average size of 4.71 nm for cNBs, the average bilayer thickness of plain LPs was 35.97 Å, and the cNB shell had an average thickness of 56.43 Å. These values agreed with the cryo-TEM measurements. Moreover, no distinct peak was evident in the low q region, indicating that cNBs did not form micelles.

Summarizing the findings, cNB-LP²⁰⁰⁰ was selected for subsequent studies. The average size of fresh cNB-LP²⁰⁰⁰ was 117.7 nm, and the size of cryopreserved ones increased to 142.7 nm over 60 days (FIGS. 22A-22C). No significant size differences were observed in cNB-LP²⁰⁰⁰ stored at 4° C. for over ten months, and pH had no significant effect on size of cNB-LP²⁰⁰⁰ during a 30-h period at 37° C. (FIGS. 22D-22E). These findings demonstrated the stability of cNB-LP²⁰⁰⁰.

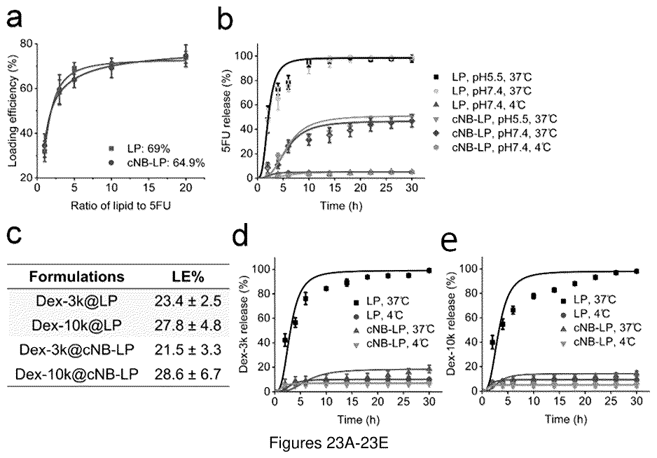

FIG. 23A shows loading efficiency of 5FU to liposome and cNB-LP as a function of lipid quantity, respectively. Five biological replicates were measured (n=5, mean value±SD).

FIG. 23 B shows in vitro release of 5FU from liposome and cNB-LP²⁰⁰⁰ at various pH levels and temperatures, respectively. Five biological replicates were measured (n=5, mean value±SD).

FIG. 23C shows loading efficiency (LE) of dextran-3k and dextran-10k to liposome and cNB-LP²⁰⁰⁰, respectively (n=5).

FIG. 23D shows in vitro release of dextran-3k and dextran-10k from liposome and cNB-LP²⁰⁰⁰ at 4° C. Five biological replicates were measured (n=5, mean value±SD)

FIG. 23E shows in vitro release of dextran-3k and dextran-10k from liposome and cNB-LP²⁰⁰⁰ at 37° C. Five biological replicates were measured (n=5, mean value±SD) [0336]5-Fluorouracil (5FU) was used. The loading efficiency increased with the lipid-to-5FU ratio, reaching a maximum of ~75% at a ratio of 20 (FIG. 23A). No significant difference in loading efficiency was identified between LPs and cNB-LP²⁰⁰⁰. A ratio of 5 was selected, achieving loading efficiency of 64.9% in cNB-LP²⁰⁰⁰. After a 14-hour incubation at 37° C., over 95% of the loaded 5FU was released from LPs (FIG. 23B). In contrast, cNB-LP²⁰⁰⁰ released ~46% and ~50% after 30 h at pH 7.4 and pH 5.5, respectively. Moreover, ~94% of the initially encapsulated 5FU remained within the core of LPs and cNB-LP²⁰⁰⁰ after 30 hours at 4° C., and
86% was retained after 10-month storage at 4° C. In addition, the loading efficiency of dextran-3k and dextran-10k in cNB-LP²⁰⁰⁰ averaged 21.5% and 28.6%, respectively, matching reported values obtained with LPs.33, 34, 35 At 37° C., LPs released over 95% of wrapped dextran after 26-h incubation, but well retained them at 4° C. Conversely, dextran were preserved in cNB-LP²⁰⁰⁰, and elevated temperature induced slightly more release (FIGS. 23C-23D).

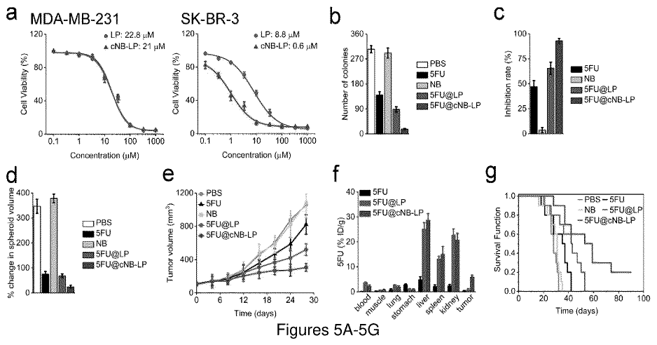

FIG. 5A shows respective IC₅₀ values of 5FU-loaded LPs and 5FU-loaded cNB-LP in treatment of MDA-MB-231 cells and SK-BR-3 cells (n=12, mean value±SD).

FIG. 5B shows growth inhibition on SK-BR-3 colonies in the respective group (n=5, mean values shown; p<0.001, one-way ANOVA).

FIG. 5C shows inhibition of migration of SK-BR-3 cells in the respective group (n=12, mean value±SD; p<0.001, one-way ANOVA).

FIG. 5D shows volume changes in the SK-BR-3 spheroids in the respective group (n=20, mean value±SD; p<0.001, one-way ANOVA).

FIG. 5E shows tumor volume of SK-BR-3 orthotopic tumor xenograft in mice after drug or placebo administration.

FIG. 5F shows biodistribution of 5FU in major organs at 24-h post-administration. Five biological replicates were measured (n=5, mean values were shown; p<0.001, one-way ANOVA).

FIG. 5G shows survival curves for mice bearing SK-BR-3 orthotopic tumor after drug or placebo administration (Ten mice in each group).

FIG. 20A shows fluorescence images and quantified data depicting the fluorescence intensity for each group illustrate the binding of PKH26-labeled cNB-LPs to HER2-overexpressed SK-BR-3 cells as opposed to HER2-deficient MDA-MB-231 cells (n=100, mean value±SD; p<0.001, one-way ANOVA).

FIG. 20B shows confocal images illustrate the binding of cNB-LPs stained with AF488-labeled anti-His6 tag antibodies to two cancer cell lines. Scale bar is 10 μm. Experiments were repeated five times.

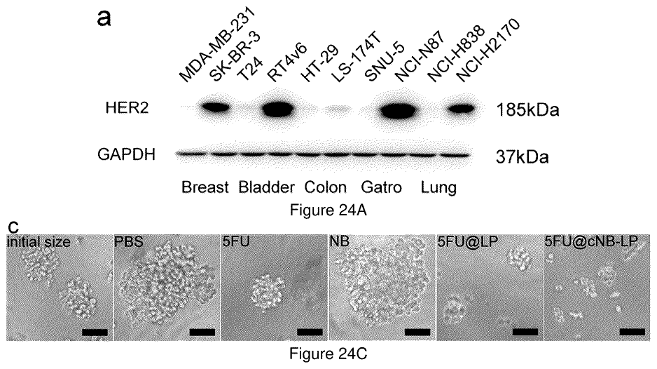

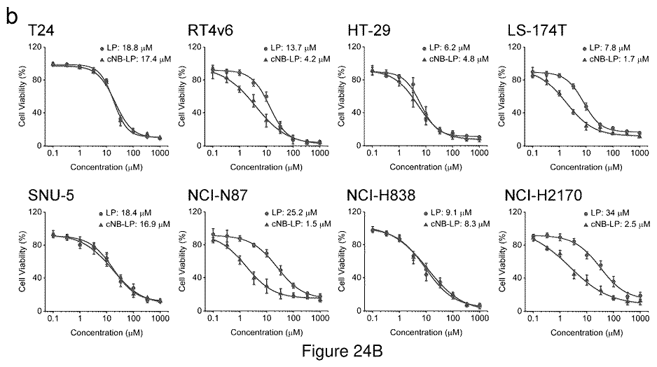

FIG. 24A shows western blot analysis of HER2 and GAPDH extracted from ten cancer cell lines.

FIG. 24B shows respective IC50 values of 5FU loaded liposomes and 5FU loaded cNB-LP in treatment of various cancer cells. Five biological replicates were measured (n=5, mean value±SD)

FIG. 24C shows the morphology of SK-BR-3 spheroids in respective groups before and after seven-day treatment. Scale bar: 100 μm. Experiments were repeated five times.

Next, HER2-overexpression cell lines and counterparts were tested (FIG. 24A). The IC50 of 5FU-loaded cNB-LP²⁰⁰⁰ in treating SK-BR-3 cells was 0.6 μM, compared to 8.8 μM for 5FU-loaded LPs (FIG. 5A), demonstrating a 14.7-fold enhancement. In the treatment of MDA-MB-231 cells, the IC50 of two groups showed no significant change. Similar results were observed in eight other cell lines (FIG. 24B). Treatment efficacy improved by 3.3- to 13.6-fold, depending on the HER2 level and sensitivity to 5FU in respective cell line. Additional cell assays demonstrated that cNB-LP²⁰⁰⁰ improved treatment efficacy in comparison with controls. The inhibitory efficacy of cNB-LP²⁰⁰⁰ in colony formation was 8.2- and 5.2-fold higher than that of free 5FU and 5FU-loaded LPs (FIG. 5B). The inhibition rate of cNB-LP²⁰⁰⁰ in cell invasion was 2- and 1.4-fold higher than that of two controls (FIG. 5C). The volume of SK-BR-3 spheroid in cNB-LP²⁰⁰⁰ and LP groups decreased 14.3- and 5.1-fold (FIG. 5D, FIG. 24C), compared to that in two negative controls.

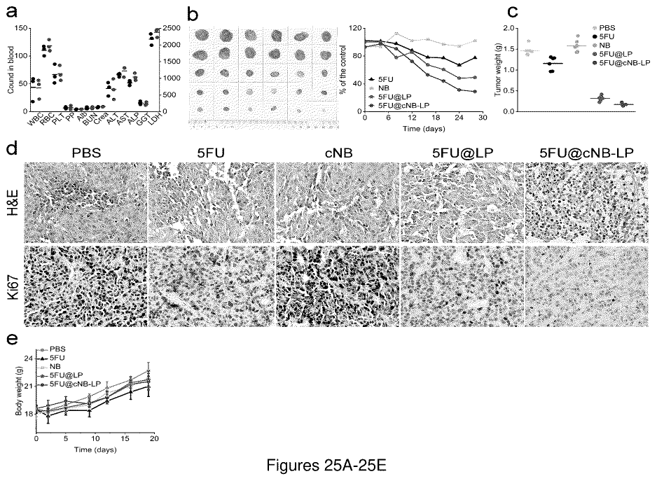

FIG. 25A shows systemic toxicity profile of free NB (n=3, three biological replicates, mean values shown; p>0.05, t-test). No significant difference in blood cell counts and blood biochemistry tests.

FIG. 25B shows SK-BR-3 tumors obtained from each group at the end of treatment and respective percentage change from baseline in tumor size (n=6, 1: PBS, 2: NB, 3: 5FU, 4: 5FU@LP, and 5: 5FU@cNB-LP).

FIG. 25C shows tumor weight of SK-BR-3 tumor in each group at the end of treatment (n=6, mean values shown; p<0.001, one-way ANOVA).

FIG. 25D shows histologic sections of tumors stained with hematoxylin/eosin and Ki67 (magnification 200×).

FIG. 25E shows average body weight of mice in each group (n=6, mean value±SD; p>0.05, one-way ANOVA).

Free NBs did not show treatment efficacy or systemic toxicity in animal studies. There was no significant difference in blood tests between PBS and NB groups (FIG. 25A). In SK-BR-3 orthotopic model, the tumor volume in cNB-LP²⁰⁰⁰ group was 3.5- and 1.7-fold smaller than that of PBS and LP groups (FIG. 5E, FIG. 25B). The tumor weight in cNB-LP²⁰⁰⁰ and LP groups was 8.6- and 4.6-fold lighter than that of PBS control (FIG. 25C). Tumor tissue damage and reduced Ki67 index were observed in the cNB-LP²⁰⁰⁰ group compared to others (FIG. 25D). Body weight showed no significant difference (FIG. 25E).

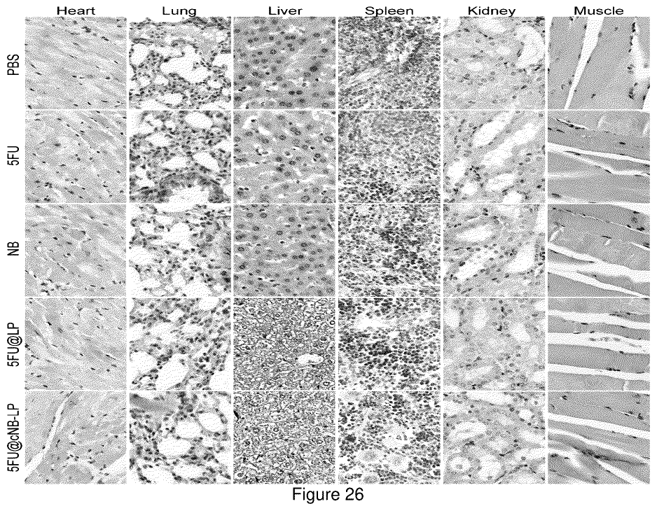

FIG. 26 shows Hematoxylin and Eosin staining of major organs and SK-BR-3 tumor in respective groups. Experiments were repeated five times (magnification 200×).

Extensive damage in major organs was not observed in any groups (FIG. 26). But histological changes were identified in the liver and spleen due to the uptake of cNB-LP²⁰⁰⁰ and LPs.

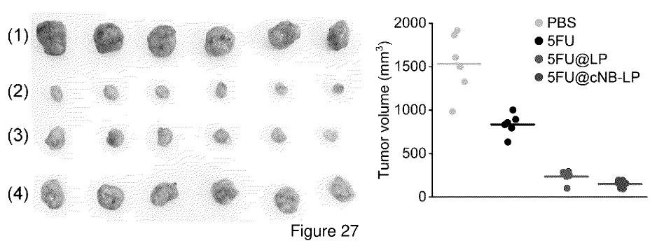

FIG. 27 shows NCI-87 tumor treatment with 5-Fluorouracil loaded chimeric nanobody decorated liposomes in vivo. NCI-N87 tumors obtained from each group at the end of treatment and respective tumor volume (1: PBS, 2: 5FU@cNB-LP, 3: 5FU@LP, and 4: 5FU; n=6, mean values shown; p<0.001, one-way ANOVA).

The biodistribution of 5FU was investigated in vivo. In cNB-LP²⁰⁰⁰ group, 5FU concentration in tumors was 18.7- and 6.9-fold higher than that in free 5FU and LP groups at 24-h post-administration (FIG. 5F). 5FU-loaded cNB-LP²⁰⁰⁰ significantly improved the median survival time of SK-BR-3 tumor-bearing mice from 28 to 56 days (FIG. 5G). Additionally, an NCI-N87 subcutaneous model was investigated. The tumor volume of NCI-N87 was 10.4-, 5.6-, and 1.6-fold smaller than that of the PBS, free 5FU, and LP groups (FIG. 27).

### Alpaca Immunization

A 3-year-old male alpaca (Vicugna pacos) was immunized with recombinant extracellular domain of human HER2. Eight subcutaneous injections were performed at 1-week intervals. Immune sera were collected at the intervals between the 4^{th} and the 5^{th} injection and the 3^{rd} day preceding the last injection. A semi-open shed was used for nighttime housing of alpaca.

### Nanobody Library Construction

Total RNA was extracted from lymphocytes followed by transcription using a SuperScript III kit (ThermoFisher, 18080093). cDNA was amplified to get nanobody (NB) coding sequences. Purified PCR products were digested with restriction enzymes and ligated with phagemid. Recombinant phagemids were transformed with TG1 cells by electroporation. The transformants were plated on LB broth solid medium supplied with ampicillin sodium.

### Screening, Expression, and Purification of NB

The obtained library was screened by phage display. Three rounds of bio-panning were carried out, and 282 colonies were selected to perform ELISA. Positive colonies were sequenced. Eventually, the candidate NB were expressed by CHO cells followed by purification.

### Antibody-Dependent Cell Cytotoxicity

Herceptin, human IgG isotype control, and NB at 15 g/ml with 5-fold serial dilutions were incubated with 2×10⁵ SK-BR-3 cells for 30 min. Peripheral blood mononuclear cells were further added and incubated at 37° C. for 15 h. Approximately 50 μl of supernatant from each well were harvested and mixed with 50 μl of LDH reagent. OD492 and OD650 were measured.

NB affinity measurement

The binding kinetics of NB to HER2 were determined using Octet RED 96e system (FortdBio, v11.0). All experiments were performed at 30° C. and reagents were prepared in 0.1% BSA, 0.02% Tween20 PBS, pH 7.4 buffer. NB was immobilized onto biosensors followed by association and dissociation measurements with human HER2 for a time window of 70 and 30 seconds, respectively.

### Capillary Isoelectric Focusing

The isoelectric point of NBs and cNBs was determined with Maurice analyzer (iCE 4.0.0). The column was filled with samples followed by applying 1.5 kV DC to pre-focus for 1 min and 3 kV DC for 7 min. The electropherogram along with the signal were recorded. Exposure time was optimized a 3 s.

### NB Induced SK-BR-3 Cell Proliferation Inhibition Assay

Herceptin, human IgG isotype, and NB at 15 μg/ml with 5-fold serial dilutions were incubated with 10,000 SK-BR-3 cells for 48 hours. Cell viability was measured followed by calculation of EC₅₀.

### Molecular Modeling Analysis

The structure of NB, HER2, NB/HER2 complex, and chimeric NB (cNB) was predicted with AlphaFold multimer (v2.3.0). GROMACS (v2021.03) was used to investigate the conformation of NB/HER2 complex.6 The force field amber14sb was used to parameterize proteins. The TIP3P was used for the waters.8 This NB/HER2 complex was solvated in a water box, and then the system was neutralized. The energy of the system was minimized. The position of heavy atoms was restricted to run constant volume equilibration and constant pressure equilibration at 300 K and 1 bar. Upon completion of the equilibration phases, a 200-ns unrestrained simulation was performed. The energy and trajectory were recorded every 20 ps. ChimeraX (v1.6.1) and Pymol (v2.5.7) were used to map interaction patterns and animate kinetic trajectories. Similarly, the cNB self-assembly was assessed.

cNB Production with E. coli

dE. coli BL21 Star™ (DE3) competent cells were transformed with recombinant plasmid ordered from GenScript. Cultures were incubated in 37° C. for 4 h and 15° C. for 16 h, respectively. Once cell density reached to OD=0.6-0.8 at 600 nm, 0.5 mM IPTG was introduced. cNBs were harvested and characterized by SDS-PAGE and Western blot.

Preparation of Liposomes and cNB-LP

DMPC, DOPE, and Cholesterol at molar ratios 700:200:100 were used to prepare liposomes. The organic solvent was evaporated using argon. Subsequently, the lipid film containing tube was placed under vacuum overnight. The lipid film was hydrated at 40° C. for 5 min. Meanwhile, cNB solution or PBS was introduced. The mixture was sonicated followed by stirring at 40° C. overnight. An Avanti Mini-Extruder equipped with a 0.1-micron polycarbonate membrane was used to prepare 100-nm LPs and cNB-LPs. Free cNBs were removed using a 100 KDa membrane filter.

### Fluorescence Microscope

cNB-LPs were stained with anti-His tag antibodies (Santa Cruz, sc-8036 AF488, 1:500; GenScript A01800, 1:500) at 4° C. Surplus antibodies were removed with a centrifugal filter. The antibody-labeled cNB-LPs were resuspended in PBS. In cell binding assay, ~5×10⁵ of cNB-LP⁵⁰⁰ and cNB-LP¹⁰⁰⁰ were incubated with 5×10³ cells for 30 min at 37° C., and unbound cNB-LPs were removed. Fluorescence images were acquired with Olympus IX83 (v01.04.07) and Leica SP5 (v2.0.2).

### Transmission Electron Microscope

Approximately 5 μl of sample was placed on a 400-mesh grid and negatively stained. For cryo-TEM, 5 μl of sample was added to a 200-mesh grid, blotted with FEI Vitrobot before plunging into liquid ethane, and transferred to a cryo-sample holder. Samples were examined in a FEI Tecnai TEM.

### cNB Anchor Efficiency

Micro-BCA protein assay (ThermoFisher, 23235) was used to determine anchor efficiency. Various cNB-LPs were prepared as described. Free cNBs were separated twice using centrifugal filters. The concentration of free cNB was measured followed by calculation of anchor efficiency.

### Forster Resonance Energy Transfer

FRET was measured with TECAN Spark (SparkControl v1.2 SP1). To prepare cNB-LPs, 1000 μM lipid mix:10 μM NBD-PE:10 μM Rhod-PE were mixed in a glass tube to make a thin film. Subsequently, 0.015 and 0.054 μM cNB were added to prepare 1 ml of cNB-LP⁵⁰⁰ and cNB-LP⁴⁰⁰⁰. Similarly, 1000.015 μM (or 1000.054 μM) lipid mix:10 μM NBD-PE:10 μM Rhod-PE were mixed to prepare plain LPs as negative controls. In another experiment, purified cNB-LP⁵⁰⁰ and cNB-LP¹⁰⁰⁰ were incubated with both anti-His6 tag antibody (Santa Cruz, sc-8036 AF488 and AF546, 1:500). Respective donor-labeled vesicles were used to calculate the FRET efficiency in the absence and presence of the acceptor according to E %=(ID−IDA)/(ID)×100, where ID and IDA are the donor intensities of samples containing only donor-labeled vesicles and samples with both donor- and acceptor-labeled vesicles, respectively.

### Laurdan Assay

The membrane fluidity was measured with a Laurdan assay. A general polarization value, GP340, was calculated as GP340=(I440-I490)/(I440+I490), where 1440 and 1490 are emission intensities at 440 and 490 nm. The temperature ranged from 10 to 42° C.

### Differential Scanning Calorimetry

Desired concentrations of 50 μl of cNB-LPs, LPs, and cNBs were placed on aluminum pans and analyzed with TA Instrument Q200 (TRIOS 5.1.1). The temperature ranged from 10 to 60° C. with a heating rate of 10° C./min.

### Circular Dichroism

CD data was obtained using a JASCO J-1500 (Spectra Manager II, v2.8) equipped with a 1-cm optical path length cell at 25° C. Approximately 0.01 mM cNBs, LPs, and cNB-LP²⁰⁰⁰ in PBS were measured, respectively.

### Atomic Force Microscopy

Samples were deposited on the mica surface. Scanning was performed using a Bruker Dimension Icon AFM (AutoMET) in contact mode with a scan rate of 1 Hz, resolution of 512×512 pixels, scanning angle of 0, and a set point below 0.1 nN. The force curve was obtained by indenting centrally an attached vesicle with a maximum force of 1.5 nN and a rate of 0.145 μm/s. The Young's modulus was extracted using PeakForce Quantitative Nanomechanical Mapping.

FIG. 18A shows low-resolution AFM scans of free LPs and cNB-LPs adsorbed onto mica substrate at room temperature. Scale bare applies to all images, 500 nm. Experiments were repeated three times.

FIG. 18B shows typical AFM images in high resolution for measurement of rigidity. Scale bar applies to all images, 200 nm. Experiments were repeated three times.

### Small-Angle X-Ray Scattering

The SAXS data was acquired with the Rigaku BioSAXS2000nano. The cNBs, LPs, and cNB-LP²⁰⁰⁰ in PBS were loaded into a quartz flow-cell mounted on a stage maintained at 4° C., aligned with the X-ray beam, respectively. A series of three 10-minute scattering images were collected. The collected data was processed using the Rigaku SAXSLab. Solvent envelopes were computed through DENSS.

### Calculation of Membrane Stiffness

A 2D model of lipid bilayer with inserted cNB (FIGS. 17A and 17B) was used. The outer radius and the thickness of the liposome are R and h, respectively. The inner radius of the liposome was used to establish a relation between Nₚ (the number of lipids) and Nₙ (the number of cNB). The areal packing density of the lipids was ρ=π/(2\sqrt{3}) (FIG. 17C), where rₚ is the radius of the lipid head. The total inner surface area of the liposome is ρA=NₚAₚ+NₙAₙ, where

$$A = 4 {π ( R - h )}^{2} , {A}_{p} = π {r}_{p}^{2} , {A}_{n} = π {r}_{n}^{2} ,$$

and rₙ is the radius of a cNB. This equation calculates Nₚ. The number of lipids surrounding a lipid is nₚ=6, whereas the average number of lipids surrounding a cNB is nₙ=π/arcsin(rₚ/(rₚ+rₙ)) (FIG. 17D). The force interaction between two adjacent lipids was approximated by a spring of constant kₚ (FIG. 17E). These springs around a molecule were in parallel under the membrane tension. Therefore, the effective stiffness due to lipid-lipid interaction was nₚkₚ. Similarly, the effective stiffness due to cNB-lipid interaction was nₙkₙ, where kₙ is the spring constant between a cNB and a lipid molecule (FIG. 17F). The effective stiffness of the membrane is k=[(nₚkₚ)(NₚAₚ)+(nₙkₙ)(NₙAₙ)]/(NₚAₚ+NₙAₙ). In the absence of cNB, the membrane stiffness is k₀=nₚkₚ. The effective stiffness of the membrane was normalized by k₀ and a relative stiffness of kᵣ=k/k₀. If kᵣ>1 obtained, the effective stiffness of the membrane increased with added cNB. Otherwise, adding cNB would reduce the membrane stiffness. Structure stability required Nₙ/Nₚ<<1, i.e., Nₙ<<4ρ/(1+(rₙ/rₚ)²)((R−h)/rₚ)². The typical geometrical parameter of rₚ, rₙ, h, and R are 0.45 nm, 0.6 nm, 5 nm, and 50 nm, respectively. With these parameters, the structure stability condition required Nₙ<<13,000. The number of cNB used in the experiments satisfied this condition. The model prediction of the effective membrane stiffness as a function of the number of cNB was plotted for different ratios of κ=kₙ/kₚ(FIG. 17G). In order for the nanobodies to enhance the stiffness of the membrane, κ>0.89 should be satisfied. The ratio κ was estimated. A generic repulsion-attraction model was used for the intermolecular interactions (FIG. 17H). The bonding energy was E=−A₁r^{-α}^{₁}+A₂r^{-α}^{₂}, where r is the intermolecular distance, α₂>α₁>0, and A₁>A₂>0. α₁ and α₂ reflect the nature of the bonding energy. These two values were assumed to be the same for lipid-lipid interaction and cNB-lipid interaction. However, A₁ and A₂ are molecule specific. At the equilibrium distance r=r₀, the minimum energy is E₀. The bonding force is F=dF/dr. The effective spring constant k is proportional to the slope of the bonding force, i.e.,

$$\begin{matrix}k ∝ dF / dr {\vert}_{r = {r}_{0}} = - [ ( {α}_{1} {α}_{2} ) / {r}_{0}^{2} ] {E}_{0} . \\ Therefore , κ = ( {E}_{0 , n} / {E}_{0 , p} ) ( {r}_{0 , p}^{2} / {r}_{0 , n}^{2} ) .\end{matrix}$$

In our problem, r_{0,n}≈r_{0,p} and E_{0,n}/E_{0,p}≈1.15. Then κ≈1.15, indicating that adding cNB increased the effective stiffness of the liposome membrane (FIG. 17G).

Computational model of interaction between cNB-LP and HER2-expression cells

A 20-μm cancer cell and a 100-nm cNB-LP were used for computation. The probability of bond formation between NB and HER2 were modeled using a probabilistic kinetic formulation. The probability of cNB-LP adhesion, denoted as Pₐ, was estimated by

$${P}_{a} = {m}_{r} {m}_{l} {K}_{a}^{0} {A}_{c} exp [ \frac{λ f}{{k}_{B} T} ] ,$$

where mₗ,mᵣ represent the ligand and receptor density, respectively,

$${K}_{a}^{0}$$

refers to the association constant at zero load of the cNB-HER2 pair, while A_{c} is the contact area between cNB-LP and cancer cell surfaces, f is the force per ligand-receptor pair, and λ is the characteristic length of the ligand receptor (assumed to be 0.1 nm), k_{B}T is the thermal energy. The drag force F and torque T induced by the fluid were balanced by the force produced by the ligand receptor pairs. When the LP is in point contact with the surface in a flow with a shear rate of γ′, the force and torque are approximated as F≈10πr²μγ′ and T≈12πr³μγ′ respectively, where r is the radius of the cNB-LP, μ is the fluid viscosity. Assuming that the force F is distributed uniformly among all the NB-HER2 bonds, and the torque T was shared uniformly only among the stretching bonds, the force per ligand-receptor pair was calculated as

$$f = \frac{1}{{m}_{r}} [ \frac{F}{{A}_{c}} + \frac{2 T}{{A}_{c} {r}_{c}} ] ,$$

where r_{c} is the radius of contact area. Data was plotted with MATLAB (R2022a).

### Zeta Potential Measurement

Zeta potential measurement was conducted using a Zetasizer Nano (v3.30). Approximately 10 μl of the sample in 990 μl of DI water was transferred to a Malvern Clear Zeta Potential cell. Three independent aliquots were analyzed three times.

### Nanosight Measurement

LPs and cNB-LP²⁰⁰⁰ in 200 μl of PBS were measured with Nanosight NS300 (v3.00).

### Drug Loading and Release Profiling

The amount of loaded 5FU in LPs and cNB-LP2000 was measured by high-performance liquid chromatography (HPLC, Waters Acquity v1.51). Loading efficiency was calculated by

$$\frac{total - un1oaded}{total} \times 100 \% .$$

To measure 5FU release, samples were placed in a 300 kDa dialysis membrane (Spectrum Labs). Samples were taken at various time points and analyzed. Similarly, the respective loading efficiency and release profiles of dextran-3k (Krackeler, 45-DF4) and dextran-10k (Krackeler, 45-FD10S) were determined.

### Cell Culture

Cells were ordered from ATCC. The SK-BR-3 (HTB-30), MDA-MB-231 (HTB-26), T24 (HTB-4), HT-29 (HTB-38), LS-174T (CL-188), NCI-N87 (CRL-5822), NCI-H838 (CRL-5844), and NCI-H2170 cells (CRL-5928) were maintained in DMEM supplied with 10% FBS. SNU-5 (CRL-5973) and RT4 cells (HTB-2) were cultured in IMDM and McCoy's 5A medium supplied with 10% FBS.

### Western Blot

Samples were electrotransferred onto a nitrocellulose membrane. The membrane was blocked with nonfat milk in PBS/0.05% Tween20 for 30 min and washed thrice with DI water. Samples were incubated overnight at 4° C. with HRP-labeled antibodies against His6 tag (Santa Cruz, sc-8036, 1:500), HER2 (Santa Cruz, sc-08, 1:500) and GAPDH (Santa Cruz, sc-32233, 1:500). Samples were washed with PBS/0.05% Tween 20 for 10 min thrice before imaging.

CCK8 assay [0415]5FU loaded LPs and cNB-LP²⁰⁰⁰ in a series of concentrations were incubated with 4,000 SK-BR-3 cells. Five replicates were made for each measurement. Cells were cultured for 24 h at 37° C. followed by incubated with 10 μl of CCK-8 for 2 h. The absorbance at 450 nm was measured.

### Colony Formation Assay

Approximately 500 SK-BR-3 cells were seeded into a well in a 6-well plate in triplicate, treated with PBS, 5FU, NB, 5FU-loaded LPs, and 5FU-loaded cNB-LP²⁰⁰⁰ for 2 weeks until visible clones were formed. The formed clones were rinsed with PBS thrice followed by numeration.

### Invasion Assay

A 24-well Transwell chamber with a pore size of 8 m was used. Each insert was coated with 50 μl of Matrigel (1:3 dilution, Corning, 354234). Approximately 1×10⁴ SK-BR-3 cells in 300 μl of medium were transferred to the upper Matrigel chamber and incubated for 48 h. Medium supplemented with 15% FBS was used as a chemoattractant and added to the lower chamber. Cells that passed through the filter were counted.

### SK-BR-3 Spheroid Assay

Round bottom Cellstar Cell-Repellent Surface plates were used. Approximately 1×10⁴ SK-BR-3 cells in 100 μl of culture medium containing 3.5% Matrigel was added to wells. When diameter reached ~100 μm, spheroids were treated with PBS, 5FU, NB, 5FU-loaded LPs, and 5FU-loaded cNB-LP²⁰⁰⁰ for 7 days. The spheroid volume were measured.

### Blood Tests

Five BALB/c mice (~18-22 g, 6 weeks) were intravenously administrated with 5 mg/kg NB for 4 doses. Approximately 250 μl of peripheral blood was collected and analyzed with Mindray BC-6200 and Beckman Coulter AU480 (v1.72).

### Animal Models

Approximately 2×10⁶ SK-BR-3 cells were inoculated to the mammary pad of BALB/c mice (~18-22 g, 6 weeks). The housing conditions for the mice were as follows: 12:12 h dark/light cycle, ambient temperature of 22±1° C., and ~55% of humidity. Tumors were allowed to grow to a size of ~100 mm3. The mice were then randomly divided into 5 groups. Drug was intravenously administrated every 2-3 days (20 mg of 5FU-equivalent per kg of body weight per dose) for 4 weeks. Mice were euthanized to harvest tumors. To study pharmacokinetics and biodistribution of 5FU, 0.1-0.3 g tissue samples were collected at 24-h post-administration followed by HPLC analysis. In parallel, 2×10⁶ NCI-N87 cells were subcutaneously inoculated to the flanks of BALB/c mice (~18-22 g, 6 weeks). Drug was intravenously administered every 2-3 days (50 mg of 5FU-equivalent per kg of body weight per dose) for 4 weeks. Moreover, SK-BR-3 orthotropic models were used to study the overall survival of mice. Fifty female BALB/c mice ζ-18-22 g, 6 weeks) were randomly divided into 5 groups and intravenously injected with 20 mg of 5FU-equivalent per kg of body weight per dose. Tumor growth, physical well-being, and survival were monitored up to 90 days. At day 90, surviving mice were sacrificed.

### Statistical Analysis

Quantitative results were presented as mean±SD. Student's unpaired t-test was used to compare control treated samples against experimental samples. For multiple treated groups, statistical significance was examined using one-way ANOVA. The sample size was not calculated before experiments. It was determined by the number of biological replicates necessary for ensuring statistical significance. Data collection and analysis were not performed blind to the conditions of the experiments. No animals for data points were excluded from the analysis.

What is claimed is:

1. An engineered nanobody conjugate, comprising:
a nanobody;
a peptide transmembrane domain or a peptide fatty acylation signal; and
a peptide linker between the nanobody and the peptide transmembrane domain.

2. The engineered nanobody conjugate of claim 1, wherein the nanobody comprises a Variable Heavy domain of Heavy chain (VHH) nanobody.

3. The engineered nanobody conjugate of claim 1, wherein the peptide transmembrane domain comprises a peptide single transmembrane domain (STMD).

4. The engineered nanobody conjugate of claim 1, wherein the nanobody comprises a Variable Heavy domain of Heavy chain (VHH) nanobody which is one of an anti-HER2 nanobody, anti-EGFR nanobody, or anti-EpCAM.

5. The engineered nanobody conjugate of claim 1, further comprising a peptide affinity tag.

6. The engineered nanobody conjugate of claim 5, wherein the peptide affinity tag has the sequence SEQ ID NO: 006 HHHHHH.

7. The engineered nanobody conjugate of claim 1, wherein the peptide linker has the sequence SEQ ID NO: 007 GGGGS, which is related between 1 and 8 times.

8. The engineered nanobody conjugate of claim 1, wherein the peptide linker comprises the sequence SEQ ID NO: 009 EAAAK.

9. The engineered nanobody conjugate of claim 1, wherein the peptide transmembrane domain comprises a HER2 STMD having the sequence SEQ. ID: 001 SIISAVVGILLVVVLGVVFGILI.

10. The engineered nanobody conjugate of claim 1, wherein the nanobody is an anti-HER2 Variable Heavy domain of Heavy chain (VHH) nanobody having the sequence SEQ ID NO: 002 ESGGGSVQSGGSLRLSCAASGYNFGWYCMGWFRQAPGKEREGVASIGGSSITKYSDSV KGRFTISRDNAKNTLYLQMNALKPEDAATYYCAARPEYDCDSLREAGWRYWGQGTQV TVSS.

11. The engineered nanobody conjugate of claim 1, wherein the nanobody is an anti-EGFR Variable Heavy domain of Heavy chain (VHH) nanobody having the sequence SEQ ID NO: 003 EVQLVESGGGSVQTGGSLRLTCAASGRTSRSYGMGWFRQAPGKEREFVSGISWRGDST GYADSVKGRFTISRDNAKNTVDLQMNSLKPEDTAIYYCAAAAGSAWYGTLYEYDYWG QGTQVTVSS.

12. The engineered nanobody conjugate of claim 1, wherein the nanobody is an anti-EpCAM Variable Heavy domain of Heavy chain (VHH) nanobody having the sequence SEQ ID NO: 004 QVQLVQSGGGSVQGGASLRLSCAASGGERNNYCVAWFRQAPGKEREVAAISRAASGA QTTTKYYVDSVKGRFTISQDTKNTATVYLQMNSHKPEDTAYCTAKAKIYPPQCTGISRTI DYRGQGTQVTVSS.

13. The engineered nanobody conjugate of claim 1, wherein the peptide fatty acylation signal is linked to a palmitoyl group or myristoyl group.

14. The engineered nanobody conjugate of claim 1, wherein the peptide transmembrane domain is inserted into a liposome membrane wherein at least a portion of the nanobody is external to the liposome, and the liposome contains a cytotoxic agent.

15. The engineered nanobody conjugate of claim 1, wherein the engineered nanobody conjugate is linked to an acyl moiety, and the acyl moiety is inserted into a liposome membrane, wherein at least a portion of the nanobody is external to the liposome, and the liposome contains a cytotoxic agent.

16. An engineered nanobody conjugate comprising an ordered sequence of:
a peptide affinity tag,
a Variable Heavy domain of Heavy chain (VHH) nanobody,
a peptide linker,
a cysteine residue, and
a peptide fatty acylation signal.

17. The engineered nanobody conjugate of claim 16, wherein the Variable Heavy domain of Heavy chain (VHH) nanobody is one of an anti-HER2 nanobody, anti-EGFR nanobody, or an anti-EpCAM nanobody, the peptide affinity tag has the sequence HHHHHH, the peptide linker has the sequence (GGGGS)ₓ, and x is equal to or greater than 1 and less than or equal to 8, and the peptide fatty acylation signal is a palmitoylation signal having the sequence SEQ ID. NO: 005 MLCCMRRTKQ or a mysistolylation signal having the sequence SEQ ID NO: 008 MGSSKS.

18. The engineered nanobody conjugate of claim 16, wherein the cysteine residue is palmitoylated.

19. A method of making an immunoliposome composition, comprising:
mixing the engineered nanobody according to claim 1 with a liposome composition; and
forming 100 nm liposomes from the liposome composition, having between 200 to 2500 chimeric nanobodies per 100 nm liposome.

20. The method of claim 19, wherein the engineered nanobody further comprises sequence SEQ ID NO: 006 HHHHHH, the peptide linker comprises at least one sequence SEQ ID NO: 007 GGGGS, the nanobody is one of an anti-HER2 nanobody, anti-EGFR nanobody, or an anti-EpCAM nanobody, and the peptide transmembrane domain or the peptide fatty acylation signal comprises an HER2 single peptide transmembrane domain SEQ. ID: 001 SIISAVVGILLVVVLGVVFGILI.
